(19) United States
(12) Patent Application Publication
(10) Pub. No.: US 2019/0060782 A1
(43) Pub. Date: Feb. 28, 2019

(54) PURIFICATION DEVICE, PURIFICATION METHOD, MANUFACTURING DEVICE, MANUFACTURING METHOD OF CHEMICAL LIQUID, CONTAINER, AND CHEMICAL LIQUID STORAGE CONTAINER

(71) Applicant: FUJIFILM Corporation, Tokyo (JP)

(72) Inventors: Tetsuya SHIMIZU, Haibara-gun (JP); Tetsuya KAMIMURA, Haibara-gun (JP)

(73) Assignee: FUJIFILM Corporation, Tokyo (JP)

(21) Appl. No.: 16/170,100

(22) Filed: Oct. 25, 2018

Related U.S. Application Data
(63) Continuation of application No. PCT/JP2017/016270, filed on Apr. 25, 2017.

(30) Foreign Application Priority Data
Apr. 28, 2016 (JP) 2016-092038
Apr. 24, 2017 (JP) 2017-085407

(51) Int. Cl. B01D 3/32; B65D 25/14; C07C 67/54; C07C 29/84

(52) U.S. Cl. CPC B01D 3/32; C07C 29/84; C07C 67/54; B65D 25/14

(57) ABSTRACT

An object of the present invention is to provide a purification device which makes it possible to obtain a solvent with a reduced impurity content and a raw material of the solvent. Another object of the present invention is to provide a purification method, a manufacturing device, and a manufacturing method of a chemical liquid. Still another object of the present invention is to provide a container which makes it difficult for an impurity content in a chemical liquid to increase even in a case where the chemical liquid is filled into the container and stored for a predetermined period of time. Yet another object of the present invention is to provide a chemical liquid storage container. The purification device of the present invention is a purification device including a distillation column for purifying a chemical liquid, in which an interior wall of the distillation column is coated with or formed of at least one kind of material selected from the group consisting of a fluororesin and an electropolished metal material, the metal material contains at least one kind of metal selected from the group consisting of chromium and nickel, and a total content of the chromium and the nickel is greater than 25% by mass with respect to a total mass of the metal material.

### CROSS-REFERENCE TO RELATED APPLICATIONS

This application is a Continuation of PCT International Application No. PCT/JP2017/016270 filed on Apr. 25, 2017, which claims priority under 35 U.S.C. § 119(a) to Japanese Patent Application No. 2016-092038 filed on Apr. 28, 2016 and Japanese Patent Application No. 2017-085407 filed on Apr. 24, 2017. Each of the above applications is hereby expressly incorporated by reference, in its entirety, into the present application.

### BACKGROUND OF THE INVENTION

### 1. Field of the Invention

The present invention relates to a purification device, a purification method, a manufacturing device, a manufacturing method of a chemical liquid, a container, and a chemical liquid storage container.

### 2. Description of the Related Art

A treatment liquid containing a solvent is used at the time of manufacturing semiconductor devices.

In recent years, there has been a demand for further reduction of impurities such as metal components contained in the solvent. In addition, as the manufacturing of a semiconductor device at a node equal to or smaller than 10 nm has been examined, the demand has become stronger.

As a method for reducing impurities from the solvent, for example, JP2008-308500A describes “manufacturing method of high-purity butyl acetate, in which at the time of manufacturing butyl acetate by synthesizing butyl acetate from acetic acid and n-butanol in the presence of a sulfuric acid catalyst, performing distillation so as to remove low boiling point products, and then performing distillation so as to remove high boiling point products, an overhead pressure of a distillation column for the high boiling point products is controlled within a range of 50 to 700 mmHg, an overhead temperature is controlled within a range of 40° C. to 120° C., and a bottom temperature is controlled within a range of 70° C. to 130° C.”.

Furthermore, JP2015-030700A describes “manufacturing method of an ester-based solvent, including performing an esterification reaction of an alcohol and a carboxylic acid in the presence of an acid catalyst and a compound forming an azeotropic mixture with water, in which a batch-type distillation apparatus including a distillation still for reacting the alcohol with the carboxylic acid, a distillation column connected to the distillation still, and a decanter connected to the top of the distillation column is used” so as to carry out the esterification reaction by a predetermined method.

In addition, JP2009-191051A describes “manufacturing method of an ester-based solvent that is for distilling and purifying a crude liquid of an esterification reaction, which is obtained by performing an esterification reaction of an alcohol and a carboxylic acid in the presence of an acid catalyst, by using a distillation column, the manufacturing method including distilling and purifying the crude liquid of the reaction without neutralizing the crude liquid, distilling away low boiling point components, and then distilling an ester-based solvent from a side cut line provided in the middle portion of the distillation column”.

### SUMMARY OF THE INVENTION

The inventors of the present invention examined the solvent such as butyl acetate distilled by the methods described in JP2008-308500A, JP2015-030700A, and JP2009-191051A. As a result, the inventors have found that, unfortunately, the impurity content does not reach the level required for the treatment liquid currently used at the time of manufacturing semiconductors.

Furthermore, as a result of examining the solvent such as butyl acetate distilled by the methods described in JP2008-308500A, JP2015-030700A, and JP2009-191051A, the inventors of the present invention have found that, unfortunately, the impurity content in the solvent increases over time in a case where the solvent is stored in a known container.

Therefore, an object of the present invention is to provide a purification device which makes it possible to obtain a solvent with a reduced impurity content and a raw material thereof (hereinafter, these will be collectively referred to as “chemical liquid”).

Another object of the present invention is to provide a purification method, a manufacturing device, and a manufacturing method of a chemical liquid.

Still another object of the present invention is to provide a container which makes it difficult for the impurity content in the chemical liquid to increase even in a case where the chemical liquid is filled into the container and stored for a predetermined period of time.

Yet another object of the present invention is to provide a chemical liquid storage container.

In order to achieve the above objects, the inventors of the present invention conducted in-depth examinations. As a result, the inventors have found that the objects can be achieved by the following constitution.

[1] A purification device comprising a distillation column for purifying a chemical liquid, in which an interior wall of the distillation column is coated with or formed of at least one kind of material selected from the group consisting of a fluororesin and an electropolished metal material, the metal material contains at least one kind of metal selected from the group consisting of chromium and nickel, and a total content of the chromium and the nickel is greater than 25% by mass with respect to a total mass of the metal material.

[2] The purification device described in [1], in which in a case where the interior wall of the distillation column is coated with the fluororesin, and the fluororesin forms a coating layer, a water contact angle on an outermost surface of the coating layer is equal to or greater than 90°, or in a case where the interior wall of the distillation column is formed of the fluororesin, a water contact angle on an outermost surface of the interior wall of the distillation column is equal to or greater than 90°.

[3] The purification device described in [1], in which in a case where the interior wall of the distillation column is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the interior wall of the distillation column is formed of the electropolished metal material, and the metal material contains chromium and iron, a mass ratio of chromium atoms contained in a surface of the interior wall of the distillation column to iron atoms contained in the surface of the interior wall of the distillation column is 0.80 to 3.0.

[4] The purification device described in any one of [1] to [3], in which an infill is disposed in the interior of the distillation column, and the infill is coated with or formed of at least one kind of material selected from the group consisting of a fluororesin and an electropolished metal material.

[5] The purification device described in [4], in which in a case where the infill is coated with the fluororesin, and the fluororesin forms a coating layer, a water contact angle on an outermost surface of the coating layer is equal to or greater than 90° , or in a case where the infill is formed of the fluororesin, a water contact angle on an outermost surface of the infill is equal to or greater than 90°.

[6] The purification device described in [4], in which in a case where the infill is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the infill is formed of the electropolished metal material, and the metal material contains chromium and iron, a mass ratio of chromium atoms contained in a surface of the infill to iron atoms contained in the surface of the infill is 0.80 to 3.0.

[7] A purification method of a chemical liquid, comprising a step of obtaining a purified product by distilling a chemical liquid by using the purification device described in any one of [1] to [6].

[8] A manufacturing device for manufacturing a chemical liquid, comprising a reaction portion for obtaining a reactant, which is a chemical liquid, by reacting a raw material; a distillation column for obtaining a purified product by distilling the reactant; and a first transfer pipeline which connects the reaction portion and the distillation column to each other so as to transfer the reactant to the distillation column from the reaction portion, in which an interior wall of the distillation column is coated with or formed of at least one kind of material selected from the group consisting of a fluororesin and an electropolished metal material, the metal material contains at least one kind of metal selected from the group consisting of chromium and nickel, and a total content of the chromium and the nickel is greater than 25% by mass with respect to a total mass of the metal material.

[9] The manufacturing device described in [8], in which in a case where the interior wall of the distillation column is coated with the fluororesin, and the fluororesin forms a coating layer, a water contact angle on an outermost surface of the coating layer is equal to or greater than 90°, or in a case where the interior wall of the distillation column is formed of the fluororesin, a water contact angle on an outermost surface of the interior wall of the distillation column is equal to or greater than 90°.

[10] The manufacturing device described in [8], in which in a case where the interior wall of the distillation column is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the interior wall of the distillation column is formed of the electropolished metal material, and the metal material contains chromium and iron, a mass ratio of chromium atoms contained in a surface of the interior wall of the distillation column to iron atoms contained in the surface of the interior wall of the distillation column is 0.80 to 3.0.

[11] The manufacturing device described in any one of [8] to [10], in which an interior wall of the first transfer pipeline is coated with or formed of at least one kind of material selected from the group consisting of a fluororesin and an electropolished metal material.

[12] The manufacturing device described in [11], in which in a case where the interior wall of the first transfer pipeline is coated with the fluororesin, and the fluororesin forms a coating layer, a water contact angle on an outermost surface of the coating layer is equal to or greater than 90°, or in a case where the interior wall of the first transfer pipeline is formed of the fluororesin, a water contact angle on an outermost surface of the interior wall of the first transfer pipeline is equal to or greater than 90°.

[13] The manufacturing device described in [11], in which in a case where the interior wall of the first transfer pipeline is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the interior wall of the first transfer pipeline is formed of the electropolished metal material, and the metal material contains chromium and iron, a mass ratio of chromium atoms contained in a surface of the interior wall of the first transfer pipeline to iron atoms contained in the surface of the interior wall of the first transfer pipeline is 0.80 to 3.0.

[14] The manufacturing device described in any one of [8] to [13], further comprising a filling portion for filling a container with the purified product; and a second transfer pipeline which connects the distillation column and the filling portion to each other so as to transfer the purified product to the filling portion from the distillation column.

[15] The manufacturing device described in [14], in which an interior wall of the second transfer pipeline is coated with or formed of at least one kind of material selected from the group consisting of a fluororesin and an electropolished metal material.

[16] The manufacturing device described in [15], in which in a case where the interior wall of the second transfer pipeline is coated with the fluororesin, and the fluororesin forms a coating layer, a water contact angle on an outermost surface of the coating layer is equal to or greater than 90°, or in a case where the interior wall of the second transfer pipeline is formed of the fluororesin, a water contact angle on an outermost surface of the interior wall of the second transfer pipeline is equal to or greater than 90°.

[17] The manufacturing device described in [15], in which in a case where the interior wall of the second transfer pipeline is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the interior wall of the second transfer pipeline is formed of the electropolished metal material, and the metal material contains chromium and iron, a mass ratio of chromium atoms contained in a surface of the interior wall of the second transfer pipeline to iron atoms contained in the surface of the interior wall of the second transfer pipeline is 0.80 to 3.0.

[18] The manufacturing device described in any one of [14] to [17], further comprising a filter portion which is disposed in the middle of the second transfer pipeline so as to filter the purified product by using a filter.

[19] The manufacturing device described in any one of [8] to [18], in which an infill is disposed in the interior of the distillation column, and the infill is coated with or formed of at least one kind of material selected from the group consisting of a fluororesin and an electropolished metal material.

[20] The manufacturing device described in [19], in which in a case where the infill is coated with the fluororesin, and the fluororesin forms a coating layer, a water contact angle on an outermost surface of the coating layer is equal to or greater than 90°, or in a case where the infill is formed of the fluororesin, a water contact angle on an outermost surface of the infill is equal to or greater than 90°.

[21] The manufacturing device described in [19], in which in a case where the infill is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the infill is formed of the electropolished metal material, and the metal material contains chromium and iron, a mass ratio of chromium atoms contained in a surface of the infill to iron atoms contained in the surface of the infill is 0.80 to 3.0.

[22] The manufacturing device described in any one of [8] to [21], in which the reaction portion includes a reactor to which the raw material is supplied and in which a reaction proceeds, and an interior wall of the reactor is coated with or formed of at least one kind of material selected from the group consisting of a fluororesin and an electropolished metal material.

[23] The manufacturing device described in [22], in which in a case where the interior wall of the reactor is coated with the fluororesin, and the fluororesin forms a coating layer, a water contact angle on an outermost surface of the coating layer is equal to or greater than 90°, or in a case where the interior wall of the reactor is formed of the fluororesin, a water contact angle on an outermost surface of the interior wall of the reactor is equal to or greater than 90°.

[24] The manufacturing device described in [22], in which in a case where the interior wall of the reactor is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or in a case where the interior wall of the reactor is formed of the electropolished metal material, and the metal material contains chromium and iron, a mass ratio of chromium atoms contained in a surface of the interior wall of the reactor to iron atoms contained in the surface of the interior wall of the reactor is 0.80 to 3.0.

[25] A manufacturing method of a chemical liquid, comprising a reaction step of obtaining a reactant, which is a chemical liquid, by reacting a raw material; and a purification step of obtaining a purified product by distilling the reactant by using a distillation column, in which an interior wall of the distillation column is coated with or formed of at least one kind of material selected from the group consisting of a fluororesin and an electropolished metal material, the metal material contains at least one kind of metal selected from the group consisting of chromium and nickel, and a total content of the chromium and the nickel is greater than 25% by mass with respect to a total mass of the metal material.

[26] The manufacturing method of a chemical liquid described in [25], in which in a case where the interior wall of the distillation column is coated with the fluororesin, and the fluororesin forms a coating layer, a water contact angle on an outermost surface of the coating layer is equal to or greater than 90°, or in a case where the interior wall of the distillation column is formed of the fluororesin, a water contact angle on an outermost surface of the interior wall of the distillation column is equal to or greater than 90°.

[27] The manufacturing method of a chemical liquid described in [25], in which in a case where the interior wall of the distillation column is electropolished, includes a coating layer formed of the metal material, and the metal material contains chromium and iron, a mass ratio of chromium atoms contained in a surface of the coating layer to iron atoms contained in the surface of the coating layer is 0.80 to 3.0, or in a case where the interior wall of the distillation column is formed of the electropolished metal material, a mass ratio of chromium atoms contained in a surface of the interior wall of the distillation column to iron atoms contained in the surface of the interior wall of the distillation column is 0.80 to 3.0.

[28] The manufacturing method of a chemical liquid described in any one of [25] to [27], further comprising a filling step of filling a container with the purified product after the purification step.

[29] The manufacturing method of a chemical liquid described in any one of [25] to [27], further comprising a filtering step of filtering the purified product by using a filter after the purification step.

[30] The manufacturing method of a chemical liquid described in [29], in which the filter is formed of at least one kind of material selected from the group consisting of nylon, polypropylene, polyethylene, polytetrafluoroethylene, and a tetrafluoroethylene-perfluoroalkyl vinyl ether copolymer.

[31] The manufacturing method of a chemical liquid described in [29] or [30], in which in the filtering step, the purified product is filtered plural times by using different kinds of filters.

[32] The manufacturing method of a chemical liquid described in any one of [29] to [31], further comprising a filling step of filling a container with the purified product after the filtering step.

[33] The manufacturing method of a chemical liquid described in any one of [25] to [32], in which the chemical liquid is used as at least one kind of agent selected from the group consisting of a pre-wet liquid, a developer, and a rinsing liquid for manufacturing a semiconductor.

[34] A container for storing a chemical liquid, in which an interior wall of the container is coated with or formed of at least one kind of material selected from the group consisting of a polyolefin resin, a fluororesin, a metal material, and an electropolished metal material, the metal material contains at least one kind of metal selected from the group consisting of chromium and nickel, and a total content of the chromium and the nickel is greater than 25% by mass with respect to a total mass of the metal material.

[35] The container described in [34], in which in a case where the interior wall of the container is coated with at least one kind of resin material selected from the group consisting of a polyolefin resin and a fluororesin, and the resin material forms a coating layer, a water contact angle on an outermost surface of the coating layer is equal to or greater than 90°, or in a case where the interior wall of the container is formed of the resin material, a water contact angle on an outermost surface of the interior wall of the container is equal to or greater than 90°.

[36] The container described in [34], in which the material is the electropolished metal material.

[37] The container described in [34] or [36], in which in a case where the metal material contains chromium and iron, a mass ratio of chromium atoms contained in a surface of the interior wall of the container to iron atoms contained in the surface of the interior wall of the container is 0.80 to 3.0.

[38] A chemical liquid storage container comprising the container described in any one of [34] to [36] and a chemical liquid stored in the container.

[39] A chemical liquid storage container described in [38], in which the chemical liquid contains metal components containing at least one kind of element selected from the group consisting of Al, Ca, Cr, Co, Cu, Fe, Pb, Li, Mg, Mn, Ni, K, Ag, Na, Ti, and Zn, and a content of metal particles, which contain the element, among the metal components is equal to or smaller than 100 mass ppt with respect to a total mass of the chemical liquid.

[40] The chemical liquid storage container described in [38], in which the chemical liquid contains metal components containing at least one kind of element selected from the group consisting of Na, K, Ca, Fe, Cr, Ti, and Ni, and a content of metal particles, which contain the element, among the metal components is equal to or smaller than 50 mass ppt with respect to a total mass of the chemical liquid.

[41] The chemical liquid storage container described in [39] or [40], in which the content of the metal particles is equal to or smaller than 10 mass ppt with respect to the total mass of the chemical liquid.

[42] The chemical liquid storage container described in any one of [38] to [41], in which the chemical liquid contains metal components containing Fe, and a content of metal particles, which contain the Fe, among the metal components is equal to or smaller than 10 mass ppt with respect to the total mass of the chemical liquid.

[43] The manufacturing method of a chemical liquid described in [28] or [32], in which in the filling step, the container described in any one of [34] to [37] is filled with the purified product.

[44] The manufacturing method of a chemical liquid described in [43], further comprising a step of washing the interior wall of the container by using a washing liquid before the filling step, in which a contact angle of the washing liquid with respect to the interior wall is 10° to 120°.

[45] The manufacturing method of a chemical liquid described in [44], in which the chemical liquid contains at least one kind of component selected from the group consisting of water and an organic solvent, and the washing liquid is at least one kind of liquid selected from the group consisting of the chemical liquid, the organic solvent, the water, and a mixture of these.

According to the present invention, a purification device which makes it possible to obtain a chemical liquid with a reduced impurity content can be provided. Furthermore, according to the present invention, a purification method, a manufacturing device, and a manufacturing method of a chemical liquid can be provided.

According to the present invention, it is possible to provide a container which makes it difficult for an impurity content in a chemical liquid to increase even in a case where the chemical liquid is filled into the container and stored for a predetermined period of time. Furthermore, according to the present invention, a chemical liquid storage container can be provided.

### BRIEF DESCRIPTION OF THE DRAWINGS

FIG. 1 is a schematic view showing a purification device according to an embodiment of the present invention.

FIG. 2 is a schematic view showing a manufacturing device according to an embodiment of the present invention.

### DESCRIPTION OF THE PREFERRED EMBODIMENTS

Hereinafter, the present invention will be specifically described based on embodiments.

The following constituents will be described based on typical embodiments of the present invention in some cases, but the present invention is not limited to the embodiments.

In the present specification, a range of numerical values described using “to” means a range including the numerical values listed before and after “to” as a lower limit and an upper limit respectively.

In the present specification, “ppm” means “parts-per-million (10⁻⁶)”, “ppb” means “parts-per-billion (10⁻⁹)”, “ppt” means “parts-per-trillion (10⁻¹²)”, and “ppq” means “parts-per-quadrillion (10⁻¹⁵)”.

### [Purification Device]

The purification device according to an embodiment of the present invention is a purification device including a distillation column for purifying a chemical liquid. The interior wall of the distillation column is coated with or formed of at least one kind of material selected from the group consisting of a fluororesin and an electropolished metal material. The metal material contains at least one kind of metal selected from the group consisting of chromium and nickel, and the total content of the chromium and the nickel is higher than 25% by mass with respect to the total mass of the metal material.

The inventors of the present invention reexamined the whole manufacturing process of a chemical liquid, and made an attempt to develop a manufacturing method of a chemical liquid with a reduced impurity content.

The inventors focused attention on a fact that in a case where a chemical liquid is purified using a purification device including a distillation column, the interior wall of the distillation column repeatedly contacts vapor, reactants, condensates, and the like. As a result, the inventors came up with an idea that a chemical liquid with a reduced impurity content may be obtained by suppressing the elution of metal components from the interior wall. Based on the idea, the inventors have found that the aforementioned objects can be achieved by a purification device using a distillation column whose interior wall is coated with or formed of a predetermined material.

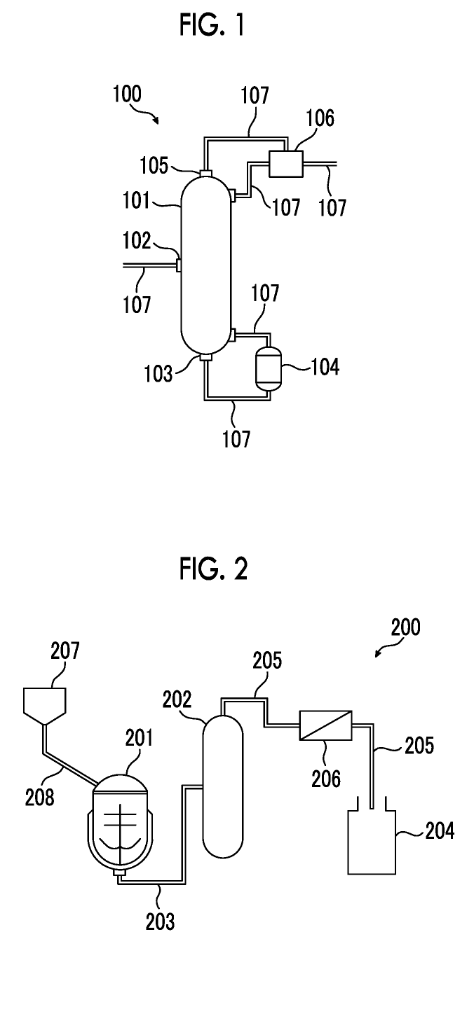

FIG. 1 is a schematic view showing the constitution of a purification device 100. The purification device 100 includes a distillation column 101 for bringing a gas and a liquid into counter-current contact to each other in the column, a supply port 102 which is connected to the distillation column 101 so as to supply a substance to be distilled to the distillation column 101, a bottom product outlet 103 provided below the supply port 102, a reboiler 104 to which the bottom product is supplied from the outlet 103 and which generates vapor by heating the supplied bottom product and supplies the vapor to the distillation column, a vapor outlet 105 provided above the supply port 102, and a condenser 106 to which the vapor taken out of the distillation column 101 is supplied from the outlet 105, which generates a condensate by cooling the supplied vapor and lets a portion of the condensate flow back to the distillation column 101, and from which the remaining condensate is taken as a purified product. Furthermore, these portions are in communication with each other through a transfer pipeline 107.

In a case where a substance to be distilled is distilled using the purification device 100, each of these portions operates as below.

First, in the interior of the distillation column 101, a portion of the substance to be distilled supplied from the supply port 102 is heated, and hence vapor is generated. The vapor is supplied to the condenser 106 from the outlet 105 and becomes a condensate. A portion of the condensate is refluxed and returns into the distillation column 101. While moving down inside the distillation column 101, a portion of the substance to be distilled supplied from the supply port 102 and the refluxed condensate contact vapor and are heated, and as a result, a portion thereof evaporates again. The liquid that has not evaporated is supplied to the reboiler 104 from the outlet 103 and returns to the distillation column 101 as vapor. The contact between the gas and the liquid are repeated by a series of processes described above, and then the purified product purified at a predetermined concentration is discharged out of the purification device 100 from the condenser 106.

In the purification device 100, the interior wall of the distillation column 101 is coated with or formed of at least one kind of the following material selected from the group consisting of a fluororesin and an electropolished metal material. Accordingly, in the process of distilling the substance to be distilled, metal components do not easily flow into the chemical liquid from the distillation column 101. Presumably, for this reason, a chemical liquid with a reduced impurity content can be obtained.

In the present specification, “coated” means that the interior wall is covered with the aforementioned material. In the aspect in which the interior wall is covered with the aforementioned material, a proportion of the total surface area of the interior wall coated with the aforementioned material is preferably equal to or higher than 70%, more preferably equal to or higher than 80%, and even more preferably equal to or higher than 90%. It is particularly preferable that the entire surface area of the interior wall is covered with the aforementioned material.

### [Material (Corrosion Resistant Material)]

The material (corrosion resistant material) is at least one kind of material selected from the group consisting of a fluororesin and an electropolished metal material.

### <Electropolished Metal Material (Metal Material Having Undergone Electropolishing)>

The metal material used for manufacturing the electropolished metal material is not particularly limited as long as it is a metal material which contains at least one kind of metal selected from the group consisting of chromium and nickel and in which the total content of the chromium and the nickel is greater than 25% by mass with respect to the total mass of the metal material. Examples thereof include stainless steel, a nickel-chromium alloy, and the like.

The total content of the chromium and the nickel in the metal material with respect to the total mass of the metal material is preferably equal to or greater than 25% by mass, and more preferably equal to or greater than 30% by mass.

The upper limit of the total content of the chromium and the nickel in the metal material is not particularly limited, but is preferably equal to or smaller than 90% by mass in general.

As the stainless steel, known stainless steel can be used without particular limitation. Among these, an alloy with a nickel content equal to or higher than 8% by mass is preferable, and austenite-based stainless steel with a nickel content equal to or higher than 8% by mass is more preferable. Examples of the austenite-based stainless steel include Steel Use Stainless (SUS) 304 (Ni content: 8% by mass, Cr content: 18% by mass), SUS304L (Ni content: 9% by mass, Cr content: 18% by mass), SUS316 (Ni content: 10% by mass, Cr content: 16% by mass), SUS316L (Ni content: 12% by mass, Cr content: 16% by mass), and the like.

Each of the Ni content and the Cr content in the above parenthesis is a content ratio with respect to the total mass of the metal material.

As the nickel-chromium alloy, known nickel-chromium alloys can be used without particular limitation. Among these, a nickel-chromium alloy is preferable in which the nickel content is 40% to 75% by mass and the chromium content is 1% to 30% by mass with respect to the total mass of the metal material.

Examples of the nickel-chromium alloy include HASTELLOY (tradename, the same is true for the following description), MONEL (tradename, the same is true for the following description), INCONEL (tradename, the same is true for the following description), and the like. More specifically, examples thereof include HASTELLOY C-276 (Ni content: 63% by mass, Cr content: 16% by mass), HASTELLOY C (Ni content: 60% by mass, Cr content: 17% by mass), HASTELLOY C-22 (Ni content: 61% by mass, Cr content: 22% by mass), and the like.

Furthermore, if necessary, the nickel-chromium alloy may further contain boron, silicon, tungsten, molybdenum, copper, cobalt, and the like in addition to the aforementioned alloy.

As the method for electropolishing the metal material, known methods can be used without particular limitation. For example, it is possible to use the methods described in paragraphs [0011] to [0014] in JP2015-227501A, paragraphs [0036] to [0042] in JP2008-264929A, and the like.

Presumably, in a case where the metal material is electropolished, the chromium content in a passive layer on the surface thereof may become higher than the chromium content in the parent phase. Presumably, for this reason, from the distillation column 101 whose interior wall is coated with or formed of the electropolished metal material, metal components may not easily flow into a chemical liquid, and hence a chemical liquid with a reduced impurity content can be obtained.

The metal material may have undergone buffing. As the buffing method, known methods can be used without particular limitation. The size of abrasive grains used for finishing the buffing is not particularly limited, but is preferably equal to or smaller than #400 because such grains make it easy to further reduce the surface asperity of the metal material.

The buffing is preferably performed before the electropolishing.

In a case where the interior wall of the distillation column is coated with the metal material having undergone electropolishing, and the metal material having undergone electropolishing forms a coating layer and contains chromium and iron, a mass ratio (Cr/Fe) of chromium (Cr) atoms contained in the surface of the coating layer to iron (Fe) atoms contained in the surface of the coating layer is not particularly limited. However, in view of obtaining a chemical liquid with a reduced impurity content, Cr/Fe is preferably equal to or higher than 0.60, more preferably equal to or higher than 0.80, even more preferably equal to or higher than 1.0, particularly preferably equal to or higher than 1.5, and most preferably higher than 1.5. Furthermore, Cr/Fe is preferably equal to or lower than 3.5, more preferably equal to or lower than 3.2, even more preferably equal to or lower than 3.0, and particularly preferably less than 2.5.

In a case where Cr/Fe is 0.80 to 3.0, a chemical liquid with a further reduced impurity content is obtained.

In a case where the interior wall of the distillation column is formed of the metal material having undergone electropolishing, and the metal material having undergone electropolishing contains chromium and iron, a mass ratio (Cr/Fe) of Cr atoms contained in the surface of the interior wall of the distillation column to Fe atoms contained in the surface of the interior wall of the distillation column is not particularly limited. However, in view of obtaining a chemical liquid with a further reduced impurity content, Cr/Fe is preferably equal to or higher than 0.60, more preferably equal to or higher than 0.80, even more preferably equal to or higher than 1.0, particularly preferably equal to or higher than 1.5, and most preferably higher than 1.5. Furthermore, Cr/Fe is preferably equal to or lower than 3.5, more preferably equal to or lower than 3.2, even more preferably equal to or lower than 3.0, and particularly preferably less than 2.5.

In a case where Cr/Fe is 0.80 to 3.0, a chemical liquid with a further reduced impurity content is obtained.

In the present specification, “surface” means a region extending by a length equal to or smaller than 5 nm from an outermost surface (interface) in a thickness direction.

In the present specification, Cr/Fe in the surface described above means Cr/Fe measured by the following method.

Measurement method: X-ray photoelectron spectrometry combined with Ar ion etching

### <Measurement Condition>

X-ray source: Al—Kα
X-ray beam diameter: φ200 μm
Signal pickup angle: 45°

### <Ion Etching Condition>

Ion species: Ar
Voltage: 2 kV
Area: 2×2 mm
Speed: 6.3 nm/min (expressed in terms of SiO₂)

### <Calculation Method>

Measurement data is obtained at an interval of 0.5 nm to a depth of 5 nm from the outermost surface, Cr/Fe is calculated for each data, and the arithmetic mean thereof is calculated.

In a case where the interior wall of the distillation column is coated with the metal material having undergone electropolishing, the thickness of the coating layer is not particularly limited, but is preferably 0.01 to 10 μm in general.

The suitable aspect described above is also applied to the infill, the interior wall of a reactor, the interior wall of a transfer pipeline, and the interior wall of a container that will be described later.

### <Fluororesin>

The aforementioned fluororesin is not particularly limited as long as it is a resin (polymer) containing fluorine atoms, and known fluororesins can be used. Examples of the fluororesin include polytetrafluoroethylene, polychlorotrifluoroethylene, polyvinylidene fluoride, a tetrafluoro ethyl ene-hexafluoropropyl ene copolymer, a tetrafluoroethylene-perfluoroalkyl vinyl ether copolymer, a tetrafluoroethylene-ethylene copolymer, a chlorotrifluoroethylene-ethylene copolymer, a cyclized perfluoro (butenyl vinyl ether) polymer (CYTOP (registered trademark)), and the like.

In a case where the interior wall of the distillation column is coated with the fluororesin, and the fluororesin forms a coating layer, a water contact angle on the outermost surface of the coating layer is not particularly limited. However, in view of obtaining a chemical liquid with a further reduced impurity content, the water contact angle is preferably equal to or greater than 90°, and more preferably greater than 90°. The upper limit of the water contact angle is not particularly limited, but is preferably equal to or smaller than 150° in general, more preferably equal to or smaller than 130°, and even more preferably less than 120°.

In a case where the interior wall of the distillation column is formed of the fluororesin, a water contact angle on the outermost surface of the interior wall of the distillation column is not particularly limited. However, in view of obtaining a chemical liquid with a further reduced impurity content, the water contact angle is preferably equal to or greater than 90°, and more preferably greater than 90°. The upper limit of the water contact angle is not particularly limited, but is preferably equal to or smaller than 150° in general, more preferably equal to or smaller than 130°, and particularly preferably less than 120°.

In the present specification, the water contact angle means a contact angle measured by the method described in Examples.

Furthermore, the outermost surface means the interface between the interior wall or the coating layer and air (or a chemical liquid and the like).

In a case where the interior wall of the distillation column is coated with the fluororesin, the thickness of the coating layer is not particularly limited, but is preferably 0.01 to 10 μm in general.

The suitable aspect described above is also applied to the infill, the interior wall of a reactor, and the interior wall of a transfer pipeline that will be described later.

The manufacturing method of the distillation column 101 is not particularly limited, and the distillation column 101 can be manufactured by known methods. For example, by a method of bonding a fluororesin lining to the interior wall of the distillation column formed of a metal, a resin, or the like, a method of coating the interior wall of the distillation column formed of a metal, a resin, or the like with a composition containing a fluororesin so as to form a coat, and the like, the distillation column whose interior wall is coated with the aforementioned material (corrosion resistant material) can be manufactured.

Furthermore, for example, by a method of electropolishing the interior wall of the distillation column formed of a metal material, in which the total content of chromium and nickel is greater than 25% by mass with respect to the total mass of the metal material, and the like, the distillation column whose interior wall is formed of the material (corrosion resistant material) can be manufactured.

It is preferable that an infill, which is not shown in the drawing, is disposed in the interior of the distillation column 101. As the infill, known infills can be used without particular limitation. Examples of the infill include a regular infill, an irregular infill, and the like.

In a case where an infill is disposed in the interior of the distillation column 101, it is preferable that the infill is coated with or formed of the material. In a case where the distillation column 101 in which the infill is disposed is used, a chemical liquid with a further reduced impurity content can be obtained.

The aspect of the material (corrosion resistant material) is as described above.

In a case where the aforementioned purification device is used, a chemical liquid with a reduced impurity content can be obtained. Specifically, the impurity content in the chemical liquid can be reduced by the following purification method.

### [Purification Method]

The purification method of a chemical liquid according to an embodiment of the present invention comprises a step of obtaining a purified product by distilling a chemical liquid by using the purification device.

As the chemical liquid which can be distilled using the purification device, known chemical liquids can be used without particular limitation.

### [Chemical Liquid (Chemical Liquid for Semiconductor)]

Examples of the chemical liquid (chemical liquid for a semiconductor) include a treatment liquid used in a manufacturing process of a semiconductor device including a lithography step, an etching step, an ion implantation step, a peeling step, and the like so as to treat an organic substance after each step is finished or before the next step is started. Specifically, examples of the chemical liquid include treatment liquids used as a developer, a rinsing liquid, a pre-wet liquid, a peeling liquid, and the like, and raw material solvents used for manufacturing these.

### <Aspect 1 of Chemical Liquid>

In an aspect, the aforementioned chemical liquid may be, for example, a chemical liquid containing one kind of compound (A) satisfying the following requirement (a) and metal components as impurities.

Requirement (a): a compound selected from an alcohol compound, a ketone compound, and an ester compound and contained in an amount of 90.0% to 99.9999999% by mass in the chemical liquid.

In many cases, the metal components contain at least one kind of element selected from the group consisting of Na, K, Ca, Fe, Ni, and Cr, for example. It has been considered that the metal components may be mainly derived from a catalyst and intermixed at the time of synthesizing the compound (A).

However, by the inventors of the present invention, it was revealed that the metal components are also eluted from the interior wall of the distillation column, and the eluted metal components are discharged together with vapor from the outlet on top of the distillation column and mixed into a purified product.

The content of the metal components in the chemical liquid is preferably 0.001 to 100 mass parts per billion (ppb) based on the total mass of the chemical liquid. In a case where the chemical liquid contains two or more kinds of metal components, the content of each of the metal components is preferably 0.001 to 100 mass ppb.

Provided that the content of each metal component is equal to or smaller than 100 mass ppb, in a case where the chemical liquid is used as a treatment liquid for a semiconductor, the metal component hardly remains as a nucleus of a residual component on a substrate at the time of treatment, and the metal component can be inhibited from becoming a cause of a defect.

The metal components in the chemical liquid include those present as ions (hereinafter, referred to as metal ions) and those present as particles (hereinafter, referred to as metal particles).

Regarding the aforementioned metal components, the inventors of the present invention consider that the content of the metal particles more easily becomes the cause of a defect compared to the content of the metal ions.

The content of the metal particles in the chemical liquid is preferably 1 to 100 mass ppt and more preferably 1 to 50 mass ppt based on the total mass of the chemical liquid.

In the present specification, the content of the metal particles means the total content of metal particles measured by a Single Nano Particle Inductively Coupled Plasma Mass Spectrometry (SP-ICP-MS) method.

The device used for the Single Particle Inductively Coupled Plasma Mass Spectrometry (hereinafter, simply referred to as “SP-ICP-MS” as well) method is the same as the device used for the general Inductively Coupled Plasma Mass Spectrometry (hereinafter, simply referred to as “ICP-MS” as well) method. The only difference between SP-ICP-MS and ICP-MS is the data analysis method. In SP-ICP-MS, the data analysis can be performed using commercial software.

In ICP-MS, the content of a metal component to be measured is measured irrespectively of the form of the metal component present. Accordingly, the total mass of particle-like metal and ionic metal containing a metal element to be measured is quantified as the content of a metal component.

In contrast, in SP-ICP-MS, the content of particle-like metal (metal particles) containing a metal element to be measured is measured.

The inventors of the present invention conducted an in-depth study regarding the influence of each of the ionic metal and the metal particles (nonionic metal) derived from metal atoms, which can be identified and quantified by the measurement using the SP-ICP-MS method and are contained in a treatment liquid in a chemical liquid, on a defect. As a result, the inventors have found that content of the metal particles in the chemical liquid exerts an extremely big influence on the occurrence of a defect. That is, the inventors have found that there is a correlation between the content of the metal particles in the chemical liquid and the occurrence of a defect.

The content of a metal component can be measured by the method described in Examples by using, for example, Agilent 8800 triple quadrupole inductively coupled plasma mass spectrometry (ICP-MS, for semiconductor analysis, option #200) manufactured by Agilent Technologies as a device for SP-ICP-MS. In addition to the above, for example, NexION 350S manufactured by PerkinElmer Inc and Agilent 8900 manufactured by Agilent Technologies can be used.

Generally, in the chemical liquid, the metal component is present in the form of particle-like metal or ionic metal in the chemical liquid. Accordingly, from the content of the metal component (Mt) measured by ICP-MS and the content of the particle-like metal (Mp) measured by SP-ICP-MS, the content of ionic metal (Mi) can be calculated based on the following equation.

Mi=Mt−Mp

Mt and Mp can be measured by ICP-MS and SP-ICP-MS respectively which adopt the devices and the conditions described later in Examples.

As described above, the compound (A) contained in the chemical liquid is a compound selected from an alcohol compound, a ketone compound, and an ester compound, for example. The chemical liquid may contain one kind or two or more kinds of these compounds.

Examples of the alcohol compound include an alcohol (monohydric alcohol) such as methanol, ethanol, 1-propanol, isopropanol, 1-butanol, 2-butanol, 3-methyl-1-butanol, tert-butyl alcohol, 1-pentanol, 2-pentanol, 1-hexanol, 3-methyl-3-pentanol, cyclopentanol, 2,3-dimethyl-2-butanol, 3,3-dimethyl-2-butanol, 2-methyl-2-pentanol, 2-methyl-3-pentanol, 3-methyl-2-pentanol, 3-methyl-3-pentanol, 4-methyl-2-pentanol, 4-methyl-3-pentanol, cyclohexanol, and 3-methoxy-1-butanol; a glycol-based solvent such as ethylene glycol, diethylene glycol, and triethylene glycol; a glycol ether-based solvent containing a hydroxyl group such as ethylene glycol monomethyl ether, propylene glycol monomethyl ether (PGME, alias name: 1-methoxy-2-propanol), diethylene glycol monomethyl ether, methoxymethyl butanol, ethylene glycol monoethyl ether, ethylene glycol monopropyl ether, and ethylene glycol monobutyl ether; and the like.

Examples of the ketone compound include acetone, 1-hexanone, 2-hexanone, cyclohexanone, methyl ethyl ketone, methyl isobutyl ketone, acetyl acetone, acetonyl acetone, acetyl carbinol, propylene carbonate, γ-butyrolactone, and the like. The ketone compound as the compound (A) also includes a diketone compound.

Examples of the ester compound include methyl acetate, ethyl acetate, butyl acetate, isobutyl acetate, propyl acetate, isopropyl acetate, methoxyethyl acetate, ethoxyethyl acetate, propylene glycol monomethyl ether acetate (PGMEA, alias name: 1-methoxy-2-acetoxypropane), ethylene glycol monoethyl ether acetate, ethylene glycol monopropyl ether acetate, ethylene glycol monobutyl ether acetate, methyl formate, ethyl formate, butyl formate, propyl formate, ethyl lactate, propyl lactate, ethyl carbonate, propyl carbonate, butyl carbonate, methyl pyruvate, ethyl pyruvate, propyl pyruvate, methyl acetoacetate, ethyl acetoacetate, methyl propionate, ethyl propionate, propyl propionate, isopropyl propionate, and the like.

The compound (A) may be a mixture of compounds such as isomers which have different structures but have the same number of carbon atoms. The mixture may contain only one kind of the compounds which have different structures but have the same number of carbon atoms or contain plural kinds of such compounds.

Examples of the step of obtaining a purified product by using the aforementioned purification device include an aspect in which a reactant containing the compound (A) obtained by reacting a predetermined raw material in the presence of a catalyst is introduced into the purification device as a substance to be distilled, and distilled under known conditions.

According to the purification method described above, a purification device including a distillation column whose interior wall is coated with or formed of the material is used. Therefore, the metal components are inhibited from being mixed into the purified product. Consequently, in the chemical liquid obtained by the purification method, an impurity content is reduced. In a case where the chemical liquid with a reduced impurity content is used as a treatment liquid for a semiconductor, a metal component hardly remains as a nucleus of a residual component on a substrate at the time of treatment, and an inorganic substance can be inhibited from becoming a cause of a defect.

### (Impurities and Coarse Particles)

It is preferable that the chemical liquid substantially does not contain coarse particles. The coarse particles incorporated into the chemical liquid are particles formed of dust, dirt, organic solids, inorganic solids, and the like incorporated into the raw material as impurities; or dust, dirt, organic solids, inorganic solids, and the like brought into the chemical liquid as contaminants in the process of preparing the chemical liquid. The coarse particles correspond to components present as particles without being dissolved in the finally obtained chemical liquid. The amount of the coarse particles present in the chemical liquid can be measured in a liquid phase by using a commercial measurement device used in a method for measuring particles in a liquid by a light scattering method using a laser as a light source.

### <Aspect 2 of Chemical Liquid>

In an aspect, the chemical liquid may be a chemical liquid which contains one kind or two or more kinds of metal atoms selected from the group consisting of Cu, Fe, and Zn and in which the total content of particle-like metal containing at least one kind of the aforementioned metal atoms is 0.01 to 100 mass parts per trillion (ppt) with respect to the total mass of the chemical liquid.

The metal elements selected from the group consisting of metal species (hereinafter, referred to as “target metals” or the like as well) including Cu, Fe, and Zn are incorporated into the chemical liquid as impurities. The particles containing these metal elements become defects and exert a big influence on the formation of a fine resist pattern and/or a fine semiconductor element. Therefore, it has been considered that the smaller the amount of the metal atoms contained in the chemical liquid, the better, because a defect occurs less in manufacturing a semiconductor. However, the inventors of the present invention have found that the amount of metal atoms contained in the chemical liquid is not necessarily correlated with a rate of occurrence of a defect, and there is a variation in the rate of occurrence of a defect. Particularly, this problem becomes marked during the formation of a semiconductor device of an ultrafine pattern (for example, equal to or smaller than 10 nm node) of recent years.

The total content of the particle-like metals (Cu, Fe, and Zn) in the chemical liquid according to the aforementioned aspect is preferably 0.01 to 50 mass ppt and more preferably 0.01 to 10 mass ppt with respect to the total mass of the chemical liquid.

As described above, the chemical liquid may be used as any of a developer, a rinsing liquid, an etching liquid, a washing liquid, a peeling liquid, and the like used in the manufacturing process of a semiconductor device. In an aspect, the chemical liquid is preferably used as a developer or a rinsing liquid.

In a case where the chemical liquid is used as a developer, the developer may be an alkaline developer or a developer containing an organic solvent.

In a case where the chemical liquid is used as an alkaline developer, the chemical liquid is preferably an aqueous solution containing a quaternary ammonium salt represented by tetramethylammonium hydroxide (TMAH). In addition, the chemical liquid may be an alkali aqueous solution containing an inorganic alkali, primary to tertiary amines, alcoholamine, cyclic amine, and the like.

Specifically, examples of the alkaline developer include alkaline aqueous solutions of inorganic alkalis such as sodium hydroxide, potassium hydroxide, sodium carbonate, sodium silicate, sodium metasilicate, and aqueous ammonia; primary amines such as ethylamine and n-propylamine; secondary amines such as diethylamine and di-n-butylamine; tertiary amines such as triethylamine and methyldiethylamine; alcoholamines such as dimethylethanolamine and triethanolamine; a quaternary ammonium salt such as tetramethylammonium hydroxide or tetraethylammonium hydroxide; cyclic amines such as pyrrole and piperidine; and the like. Among these, an aqueous solution of tetramethylammonium hydroxide or tetraethylammonium hydroxide is preferable.

Furthermore, alcohols and a surfactant may be added to the alkaline developer in an appropriate amount. The alkali concentration in the alkaline developer is generally 0.1% to 20% by mass. The pH of the alkaline developer is generally 10.0 to 15.0.

Development using the alkaline developer is performed generally for 10 to 300 seconds.

The alkali concentration (and pH) of the alkaline developer and the development time can be appropriately adjusted according to the pattern to be formed.

In a case where the chemical liquid is used as a developer containing an organic solvent (hereinafter, referred to as “organic developer” as well), as the organic solvent, it is possible to use a polar solvent and a hydrocarbon-based solvent such as a ketone-based solvent, an ester-based solvent, an alcohol-based solvent, an amide-based solvent, and an ether-based solvent. In the present invention, a solvent of a grade, in which inorganic ions such as sulfate ions, chloride ions, or nitrate ions or Fe, Cu, and Zn as target metals are reduced, is preferably used, or such a solvent that is further purified is preferably used.

Examples of the ketone-based solvent include 1-octanone, 2-octanone, 1-nonanone, 2-nonanone, acetone, 2-heptanone (methyl amyl ketone), 4-heptanone, 1-hexanone, 2-hexanone, diisobutyl ketone, cyclohexanone, methyl cyclohexanone, phenyl acetone, methyl ethyl ketone, methyl isobutyl ketone, acetyl acetone, acetonyl acetone, ionone, diacetonyl alcohol, acetyl carbinol, acetophenone, methyl naphthyl ketone, isophorone, propylene carbonate, and the like.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutylacetate, 3-methyl-3-methoxybutylacetate, methyl formate, ethyl formate, butyl formate, propyl formate, ethyl lactate, butyl lactate, propyl lactate, and the like.

Examples of the alcohol-based solvent include an alcohol such as methyl alcohol, ethyl alcohol, n-propyl alcohol, isopropyl alcohol (IPA), n-butyl alcohol, sec-butyl alcohol, tert-butyl alcohol, isobutyl alcohol, 4-methyl-2-pentanol (methyl isobutyl carbinol; MIBC), n-hexyl alcohol, n-heptyl alcohol, n-octyl alcohol, and n-decanol; a glycol-based solvent such as ethylene glycol, diethylene glycol, and triethylene glycol; a glycol ether-based solvent such as ethylene glycol monomethyl ether, propylene glycol monomethyl ether, ethylene glycol monoethyl ether, propylene glycol monoethyl ether, diethylene glycol monomethyl ether, triethylene glycol monoethyl ether, and methoxymethyl butanol; and the like.

Examples of the ether-based solvent include the aforementioned glycol ether-based solvent, dioxane, tetrahydrofuran, and the like.

Examples of the amide-based solvent include N-methyl-2-pyrrolidone (NMP), N,N-dimethylacetamide, N,N-dimethylformamide, hexamethyl phosphoric triamide, 1,3-dimethyl-2-imidazolidinone, and the like.

Examples of the hydrocarbon-based solvent include an aromatic hydrocarbon-based solvent such as toluene and xylene, and an aliphatic hydrocarbon-based solvent such as pentane, hexane, octane, decane, and undecane.

A plurality of solvents described above may be mixed together. Alternatively, the above solvents may be used by being mixed with solvents other than the above and/or water. Here, in order to make the effects of the present invention sufficiently exerted, the total moisture content in the developer is preferably less than 10% by mass. It is more preferable that the developer substantially does not contain moisture.

Particularly, the organic developer is preferably a developer containing at least one kind of organic solvent selected from the group consisting of a ketone-based solvent, an ester-based solvent, an alcohol-based solvent, an amide-based solvent, and an ether-based solvent.

The vapor pressure of the organic developer at 20° C. is preferably equal to or lower than 5 kPa, more preferably equal to or lower than 3 kPa, and even more preferably equal to or lower than 2 kPa. In a case where the vapor pressure of the organic developer is equal to or lower than 5 kPa, the evaporation of the developer that occurs on a substrate or in a developing cup is inhibited, and the temperature uniformity within a wafer surface is improved. As a result, the dimensional uniformity within the wafer surface becomes excellent.

If necessary, a sufficient amount of a surfactant may be added to the organic developer.

The surfactant is not particularly limited, and for example, ionic and/or nonionic fluorine-based surfactant and/or silicone-based surfactant can be used. Examples of the fluorine-based surfactant and/or silicon-based surfactant include the surfactants described in JP1987-036663A (JP-S62-036663A), JP1986-226746A (JP-S61-226746A), JP1986-226745A (JP-S61-226745A), JP1987-170950A (JP-S62-170950A), JP1988-034540A (JP-S63-034540A), JP1995-230165A (JP-H07-230165A), JP1996-062834A (JP-H08-062834A), JP1997-054432A (JP-H09-054432A), JP1997-005988A (JP-H09-005988A), U.S. Pat. Nos. 5,405,720A, 5,360,692A, 5,529,881A, 5,296,330A, 5,436,098A, 5,576,143A, 5,294,511A, and 5,824,451A. As the surfactant, a nonionic surfactant is preferable. The nonionic surfactant is not particularly limited, but a fluorine-based surfactant or a silicon-based surfactant is even more preferably used.

The amount of the surfactant used is generally 0.001% to 5% by mass, preferably 0.005% to 2% by mass, and more preferably 0.01% to 0.5% by mass, with respect to the total amount of the developer.

The organic developer is preferably butyl acetate.

The organic developer may contain a nitrogen-containing compound exemplified in paragraphs [0041] to [0063] in JP5056974B. From the viewpoint of the storage stability of the developer and the like, it is preferable that the nitrogen-containing compound is added to the organic developer immediately before the formation of a pattern.

In a case where the chemical liquid is used as a rinsing liquid, it is preferable that the chemical liquid contains an organic solvent. In the present invention, a solvent of a grade, in which inorganic ions such as sulfate ions, chloride ions, or nitrate ions or Fe, Cu, and Zn as target metals are reduced, is preferably used, or such a solvent that is further purified is preferably used.

The amount of the organic solvent used with respect to the total amount of the rinsing liquid containing an organic solvent (hereinafter, referred to as “organic rinsing liquid” as well) is preferably equal to or greater than 90% by mass and equal to or smaller than 100% by mass, more preferably equal to or greater than 95% by mass and equal to or smaller than 100% by mass, and even more preferably equal to or greater than 95% by mass and equal to or smaller than 100% by mass.

The organic rinsing liquid is not particularly limited as long as it does not dissolve a resist pattern. As the organic rinsing liquid, a solution containing a general organic solvent can be used. In a case where the chemical liquid is used as the organic rinsing liquid, it is preferable that the chemical liquid contains at least one kind of organic solvent selected from the group consisting of a hydrocarbon-based solvent, a ketone-based solvent, an ester-based solvent, an alcohol-based solvent, an amide-based solvent, and an ether-based solvent.

Specific examples of the hydrocarbon-based solvent, the ketone-based solvent, the ester-based solvent, the alcohol-based solvent, the amide-based solvent, and the ether-based solvent include the same solvents as those described above regarding the organic developer.

Particularly, the chemical liquid as an organic rinsing liquid preferably contains at least one kind of solvent selected from the group consisting of N-methyl-2-pyrrolidone (NMP), isopropyl alcohol (IPA), ethanol, and 4-methyl-2-pentanol (MIBC).

The moisture content in the organic rinsing liquid is preferably equal to or smaller than 10% by mass, more preferably equal to or smaller than 5% by mass, and even more preferably equal to or smaller than 3% by mass. In a case where the moisture content is equal to or smaller than 10% by mass, excellent developing characteristics can be obtained.

The vapor pressure of the organic rinsing liquid at 20° C. is preferably equal to or higher than 0.05 kPa and equal to or lower than 5 kPa, more preferably equal to or higher than 0.1 kPa and equal to or lower than 5 kPa, and even more preferably equal to or higher than 0.12 kPa and equal to or lower than 3 kPa. In a case where the vapor pressure of the rinsing liquid is equal to or higher than 0.05 kPa and equal to or lower than 5 kPa, the temperature uniformity within a wafer surface is improved, the swelling resulting from the permeation of the rinsing liquid is inhibited, and the dimensional uniformity within the wafer surface becomes excellent.

It is also possible to use the organic rinsing liquid by adding a sufficient amount of the aforementioned surfactant thereto.

### <Aspect 3 of Chemical Liquid>

In another aspect, the chemical liquid may be a composition (chemical liquid) which contains hydrogen peroxide, an acid, and a Fe component and in which a mass ratio of the content of the Fe component to the content of the acid is 10⁻⁵ to 10².

It is considered that the Fe component is present to a certain extent in a solvent or a raw material component including anthraquinone which will be described later, and mixed into the composition through the solvent or the raw material. In the present aspect, the Fe component includes Fe in the form of ions and Fe in the form of metal particles. Furthermore, the Fe particles include not only Fe in the form of metal particles but also colloidal Fe. That is, the Fe component means all the Fe atoms contained in the composition, and the content of the Fe component means the total amount of the metal.

In the process of preparing the aforementioned composition, the Fe component may be removed by purification such that the content thereof becomes less than the lower limit of the predetermined range of numerical values described above, and then the Fe component is added such that the content thereof becomes a predetermined range of numerical values.

In addition, the purification for removing the impurity may be performed on the solvent or the raw material component used in the process of synthesizing hydrogen peroxide or performed on the composition containing hydrogen peroxide after the synthesis of hydrogen peroxide.

The content of the Fe component in the composition is preferably 0.1 mass ppt to 1 mass ppb with respect to the total mass of the composition. In a case where the content of the Fe component in the composition is within the above range, the Fe component hardly exerts an influence on the occurrence of a defect in a semiconductor substrate.

The content of the acid in the composition is preferably 0.01 mass ppb to 1,000 mass ppb with respect to the total mass of the composition. In a case where the content of the acid is less than 0.01 mass ppb with respect to the total mass of the composition, sometimes the content of the Fe component in the composition becomes relatively too large. In a case where the content of the acid is equal to or greater than 0.01 mass ppb with respect to the total mass of the composition, the content of the Fe component is adjusted within an appropriate range. Accordingly, the storage stability is further improved, or the Fe component does not become a nucleus and forms particles in the liquid, and in a case where the chemical liquid is applied to a manufacturing process of a semiconductor device, the occurrence of a defect in a semiconductor substrate can be inhibited.

In contrast, in a case where the content of the acid is greater than 1,000 mass ppb with respect to the total mass of the composition, sometimes the content of the Fe component in the composition becomes relatively too small. In a case where the content of the acid is equal to or smaller than 1,000 mass ppb with respect to the total mass of the composition, colloidal particles are hardly formed in the liquid, and in a case where the composition is applied to a manufacturing process of a semiconductor device, the occurrence of a defect in a semiconductor substrate can be inhibited.

The hydrogen peroxide is generally synthesized by an anthraquinone method. In many cases, in the composition containing the hydrogen peroxide synthesized and obtained by the anthraquinone method, a certain amount of impurities (for example, anthraquinone compounds or metal components containing an element selected from the group consisting of Ni, Pt, Pd, and Al that are derived from a catalyst which can be used in a step of reducing anthraquinone so as to synthesize anthrahydroquinone) derived from raw materials remain, although the amount thereof is trace. Generally, it is desirable that these impurities are removed. However, it is preferable that the impurities are not completely removed from the composition but are allowed to remain in a trace amount in the composition.

The content of the anthraquinone compounds in the composition is preferably 0.01 mass ppb to 1,000 mass ppb with respect to the total mass of the composition. Provided that the content of the anthraquinone compounds with respect to the total mass of the composition is equal to or greater than 0.01 mass ppb, a defective performance can be effectively improved. In contrast, provided that the content of the anthraquinone compounds with respect to the total mass of the composition is equal to or smaller than 1,000 mass ppb, in a case where the composition is applied to a manufacturing process of a semiconductor device, the compounds hardly exert an influence on the occurrence of a defect in a semiconductor substrate.

In the composition, the content of the metal components containing an element selected from the group consisting of Ni, Pt, Pd, and Al is preferably 0.01 mass ppt to 1 mass ppb with respect to the total mass of the composition. Herein, the metal components are present in the form of metal ions or in the form of metal particles. That is, for example, in a case where the composition contains a Pt component, the content of the PT components means the total amount of the metal Pt (the total amount of the metal is as described above). Provided that the content of the metal component containing an element selected from the group consisting of Ni, Pt, Pd, and Al is equal to or greater than 0.01 mass ppb with respect to the total mass of the composition, the oxidizing power of the composition is further improved. In contrast, provided that the content of the metal components containing an element selected from the group consisting of Ni, Pt, Pd, and Al is equal to or smaller than 1,000 mass ppb with respect to the total mass of the composition, in a case where the composition is applied to a manufacturing process of a semiconductor device, the metal components hardly exert an influence on the occurrence of a defect in a semiconductor substrate. In a case where the composition contains a plurality of kinds of metal components containing an element selected from the group consisting of Ni, Pt, Pd, and Al, it is preferable that the amount of each of the metal components falls into the above range.

Hereinafter, each of the components of the composition will be more specifically described.

### (Hydrogen Peroxide)

The content of hydrogen peroxide in the composition is preferably 0.001% to 70% by mass, more preferably 10% to 60% by mass, and even more preferably 15% to 60% by mass.

### (Acid)

The composition contains an acid. The “acid” mentioned herein does not include hydrogen peroxide.

The acid is not particularly limited as long as it can adsorb metal ions present in a liquid (for example, the ions are adsorbed in the form of ionic bonding or coordinate bonding), but the acid is preferably a water-soluble acidic compound.

The water-soluble acidic compound is not particularly limited as long as it has a dissociable functional group which exhibits acidity by being dissolved in water. The water-soluble acidic compound may be an organic compound or an inorganic compound. Herein, “water-soluble” means that the compound is dissolved in an amount equal to or greater than 5 g in 100 g of water at 25° C.

Examples of the water-soluble acidic compound and a salt thereof include an acidic compound such as an inorganic acid including hydrochloric acid, sulfuric acid, phosphoric acid, nitric acid, or the like, a carboxylic acid derivative, a sulfonic acid derivative, a phosphoric acid derivative, and the like. In addition, compounds in which these acidic functional groups form salts may be adopted.

Among these, as the water-soluble acidic compound, a phosphoric acid derivative or phosphoric acid is preferable because such a compound can effectively chelate and remove impurities.

Examples of the phosphoric acid derivative include pyrophosphoric acid or polyphosphoric acid.

Examples of a cation forming a salt together with the water-soluble acidic compound include an alkali metal, an alkaline earth metal, a quaternary alkyl compound (for example, tetramethylammonium hydroxide (TMAH), tetraethylammonium hydroxide (TEAH), tetrapropylammonium hydroxide (TPAH), or tetrabutylammonium hydroxide (TBAH)), and the like. One kind of the cation forming a salt may be used singly, or two or more kinds of the cations forming a salt may be used in combination.

As the water-soluble acidic compound, in addition to the compounds described above, a so-called chelating agent may be used. The chelating agent is not particularly limited, but is preferably polyaminopolycarboxylic acid.

The polyaminopolycarboxylic acid is a compound having a plurality of amino groups and a plurality of carboxylic acid groups. Examples thereof include mono- or polyalkylene polyamine polycarboxylic acid, polyaminoalkane polycarboxylic acid, polyaminoalkanol polycarboxylic acid, and hydroxyalkylether polyamine polycarboxylic acid.

Examples of suitable polyaminopolycarboxylic acid chelating agents include butylenediaminetetraacetic acid, diethylenetriaminepentaaceic acid (DTPA), ethylenediaminetetrapropionic acid, triethylenetetraaminehexaacetic acid, 1,3-diamino-2-hydroxypropane-N,N,N′,N′-tetraacetic acid, propylenediaminetetraacetic acid, ethylenediaminetetraacetic acid (EDTA), trans-1,2-diaminocyclohexanetetraacetic acid, ethylenediamine diacetic acid, ethylenediamine dipropionic acid, 1,6-hexamethylene-diamine-N,N,N′,N′-tetraacetic acid, N,N-bis(2-hydroxybenzyl)ethylenediamine-N,N-diacetic acid, diaminopropanetetraacetic acid, 1,4,7,10-tetraazacyclododecane-tetraacetic acid, diaminopropanol tetraacetic acid, and (hydroxyethyl)ethylenediamine triacetic acid. Among these, diethylenetriaminepentaacetic acid (DTPA), ethylenediaminetetraacetic acid (EDTA), and trans-1,2-diaminocyclohexanetetraacetic acid are preferable.

In the composition, one kind of acid can be used singly, or two or more kinds of acids can be used in combination.

As described above, the content of the acid with respect to the total mass of the composition is preferably 0.01 mass ppb to 1,000 mass ppb, more preferably 0.05 mass ppb to 800 mass ppb, and even more preferably 0.05 mass ppb to 500 mass ppb.

### (Fe Component)

The composition contains an Fe component.

As described above, in the composition, a mass ratio of the content of the Fe component to the content of the acid is preferably 10⁻⁵ to 10², and more preferably 10⁻³ to 10⁻¹.

Furthermore, as described above, in the composition, the content of the Fe component with respect to the total mass of the composition is preferably 0.1 mass ppt to 1 mass ppb, more preferably 0.1 mass ppt to 800 mass ppt, and even more preferably 0.1 mass ppt to 500 mass ppt. The content mentioned herein is the content of Fe atoms.

### (Water)

The composition may contain water as a solvent.

The content of the water is not particularly limited, but may be 1% to 99.999% by mass with respect to the total mass of the composition.

As the water, ultrapure water used for manufacturing semiconductor devices is preferable. Although the water is not particularly limited, water in which the ion concentration of metal elements such as Fe, Co, Na, K, Ca, Cu, Mg, Mn, Li, Al, Cr, Ni, and Zn is reduced is preferable, and water in which the ion concentration of the metal elements is adjusted at the time of using the water for preparing the composition such that the ion concentration becomes equal to or lower than a ppt order is more preferable. As the adjustment method, purification using a filtration membrane or an ion-exchange membrane or purification by distillation is preferable. Examples of the adjustment method include the method described in paragraphs [0074] to [0084] in JP2011-110515A.

In the present specification, the water used in each embodiment is preferably the water obtained as above.

In a case where the composition is used as a chemical liquid, the water is more preferably used not only as water in the composition but also as water for washing a container, water used in a kit which will be described later, and the like.

### (Anthraquinone Compounds)

The composition may contain anthraquinone compounds.

Examples of the anthraquinone compounds include a compound obtained in a process of synthesizing hydrogen peroxide by an anthraquinone method. Specifically, as the anthraquinone compounds, at least one or more kinds of compounds selected from the group consisting of alkyl anthraquinone and alkyl tetrahydroanthraquinone are preferable.

The alkyl group contained in the alkyl anthraquinone and the alkyl tetrahydroanthraquinone preferably has 1 to 8 carbon atoms, and more preferably has 1 to 5 carbon atoms. As the alkyl anthraquinone, ethyl anthraquinone or amyl anthraquinone is particularly preferable. As the alkyl tetrahydroanthraquinone, ethyl tetrahydroanthraquinone or amyl tetrahydroanthraquinone is particularly preferable.

One kind of anthraquinone compounds can be mixed with the composition, or two or more kinds of anthraquinone compounds can be mixed in combination with the composition.

In a case where the composition contains the anthraquinone compounds, as described above, the content of the anthraquinone compounds is preferably 0.01 mass ppb to 1,000 mass ppb with respect to the total mass of the composition. From the viewpoint of further improving the effects of the present invention, the content of the anthraquinone compounds is more preferably 0.05 mass ppb to 800 mass ppb, and even more preferably 0.05 mass ppb to 500 mass ppb.

(Metal Components Containing Element Selected from Group Consisting of Ni, Pt, Pd, and Al)

The composition may contain at least one or more kinds of metal components containing an element selected from the group consisting of Ni, Pt, Pd, and Al.

In a case where the composition contains the metal components containing an element selected from the group consisting of Ni, Pt, Pd, and Al, as described above, the content of the metal components with respect to the total mass of the composition is preferably 0.01 mass ppt to 1 mass ppb, more preferably 0.01 mass ppt to 800 mass ppt, and even more preferably 0.01 mass ppt to 500 mass ppt.

The composition may contain other additives in addition to the components described above. Examples of other additives include a surfactant, an antifoaming agent, a pH adjuster, a fluoride, and the like.

### <Aspect 4 of Chemical Liquid>

In another aspect, the aforementioned chemical liquid may be a chemical liquid which contains at least one kind of organic solvent (hereinafter, referred to as “specific organic solvent” as well) selected from the group consisting of ethers, ketones, and lactones, water, and a metal components (hereinafter, referred to as “specific metal components” as well) containing at least one kind of metal element selected from the group consisting of Na, K, Ca, Fe, Cu, Mg, Mn, Li, Al, Cr, Ni, Ti, and Zn, in which the content of the water in the chemical liquid is 100 mass ppb to 100 mass ppm, and the content of the metal components in the chemical liquid is 10 mass ppq to 10 mass ppb.

By the chemical liquid according to the aforementioned aspect, the occurrence of a defect in a semiconductor device can be inhibited, and excellent corrosion resistance and wettability can be obtained.

### (Specific Organic Solvent)

The chemical liquid contains a specific organic solvent. The specific organic solvent is at least one kind of organic solvent selected from the group consisting of ethers, ketones, and lactones as described above.

One kind of specific organic solvent may be used singly, or two or more kinds of specific organic solvents may be used in combination.

In a case where the chemical liquid contains two or more kinds of specific organic solvents, the content of the specific organic solvent means the total content of the two or more kinds of specific organic solvents.

### Ethers

“Ethers” is the generic name of organic solvents having an ether bond. As the ethers, diethylene glycol dimethyl ether, tetrahydrofuran, ethylene glycol monomethyl ether, ethylene glycol monoethyl ether, methyl cellosolve acetate, ethyl cellosolve acetate, diethylene glycol monomethyl ether, diethylene glycol monoethyl ether, diethylene glycol monobutyl ether, propylene glycol monomethyl ether, propylene glycol monomethyl ether acetate, propylene glycol monoethyl ether acetate, and propylene glycol monopropyl ether acetate, and the like are preferably used.

Among the ethers, from the viewpoint of improving residues, propylene glycol monomethyl ether acetate, propylene glycol monomethyl ether, diethylene glycol monomethyl ether, diethylene glycol monoethyl ether, and diethylene glycol monobutyl ether are preferable, and propylene glycol monomethyl ether acetate, propylene glycol monomethyl ether, and diethylene glycol monobutyl ether are more preferable.

One kind of ethers may be used singly, or two or more kinds of ethers may be used in combination.

### Ketones

“Ketones” is the generic name of organic solvents having a ketone structure. As the ketones, methyl ethyl ketone (2-butanone), cyclohexanone, cyclopentanone, 2-heptanone, 3-heptanone, 4-heptanone, N-methyl-2-pyrrolidone, methyl propyl ketone (2-pentanone), methyl-n-butyl ketone (2-hexanone), methyl isobutyl ketone (4-methyl-2-heptanone), and the like are preferably used.

Among the ketones, methyl ethyl ketone, methyl propyl ketone, methyl isobutyl ketone, and cyclohexanone are preferable, and methyl ethyl ketone, methyl propyl ketone, and cyclohexanone are more preferable, because these make it possible to further improve the occurrence of a defect in a semiconductor device.

One kind of ketones may be used singly, or two or more kinds of ketones may be used in combination.

### Lactones

Lactones refer to aliphatic cyclic esters having 3 to 12 carbon atoms. As the lactones, for example, β-propiolactone, γ-butyrolactone, γ-valerolactone, δ-valerolactone, γ-caprolactone, ϵ-caprolactone, and the like are preferably used.

Among the lactones, γ-butyrolactone and γ-caprolactone are preferable, and γ-butyrolactone is more preferable, because these make it possible to further improve the occurrence of a defect in a semiconductor device.

One kind of lactones may be used singly, or two or more kinds of lactones may be used in combination.

Among these organic solvents, at least one kind of ethers are preferably used, and two or more ethers are more preferably used in combination, because then the occurrence of a defect in a semiconductor device can be further reduced.

In a case where two or more kinds of ethers are combined, as the ethers to be combined, propylene glycol monomethyl ether acetate, propylene glycol monomethyl ether, diethylene glycol monomethyl ether, diethylene glycol monoethyl ether, and diethylene glycol monobutyl ether are preferable.

Among these, a combination (mixed solvent) of propylene glycol monomethyl ether acetate and propylene glycol monomethyl ether is preferable. In this case, a mixing ratio between the propylene glycol monomethyl ether acetate and the propylene glycol monomethyl ether is preferably within a range of 1:5 to 5:1.

### (Water)

The aforementioned chemical liquid contains water. The water may be moisture which is inevitably incorporated into each component (raw material) contained in the chemical liquid, moisture which is inevitably incorporated at the time of manufacturing the chemical liquid, or intentionally added water.

The content of water in the chemical liquid is 100 mass ppb to 100 mass ppm, preferably 100 mass ppb to 10 mass ppm, and more preferably 100 mass ppb to 1 mass ppm. In a case where the content of water is equal to or greater than 100 mass ppb, the wettability of the chemical liquid becomes excellent, and the occurrence of a defect in a semiconductor device can also be inhibited. Furthermore, in a case where the content of water is equal to or smaller than 100 mass ppm, the corrosion resistance of the chemical liquid becomes excellent.

The content of water in the chemical liquid is measured by the method described in Examples, which will be described later, by using a device which adopts the Karl Fischer moisture measurement method (coulometric titration method) as the principle of measurement.

As one of the methods for making the content of water in the chemical liquid fall into the above range, for example, there is a method in which the chemical liquid is placed in a desiccator having undergone nitrogen purging and warmed in the desiccator in a state where the internal pressure of the desiccator is kept positive. Furthermore, by the method exemplified in the purification step which will be described later, the content of water in the chemical liquid can also be adjusted within a desirable range.

### (Specific Metal Components)

The chemical liquid contains specific metal components. As described above, the specific metal components are metal components containing at least one kind of metal element selected from the group consisting of Na, K, Ca, Fe, Cu, Mg, Mn, Li, Al, Cr, Ni, and Zn.

The chemical liquid may contain only one kind of the specific metal components or contain two or more kinds of the specific metal components.

The specific metal components may be in any form such as ions, a complex compound, a metal salt, an alloy, and the like. Furthermore, the specific metal components may be in the form of particles.

The specific metal components may be metal components which are unintentionally added to each component (raw material) contained in the chemical liquid, metal components which are unintentionally incorporated at the time of manufacturing the chemical liquid, or metal components which are intentionally added.

The content of the specific metal components in the chemical liquid is 10 mass ppq to 10 mass ppb, preferably 10 mass ppq to 300 mass ppt, more preferably 10 mass ppq to 100 mass ppt, and even more preferably 20 mass ppt to 100 mass ppt. In a case where the content of the specific metal components is within the above range, the occurrence of a defect in a semiconductor device can be inhibited.

In a case where the chemical liquid contains two or more kinds of the specific metal components, the aforementioned content of the specific metal components means the total content of the two or more kinds of the specific metal components.

The specific metal components in the chemical liquid may contain a particle-like specific metal component. In this case, the content of the particle-like specific metal component (metal particles) in the chemical liquid is preferably 1 mass ppq to 1 mass ppb, more preferably 1 mass ppq to 30 mass ppt, even more preferably 1 mass ppq to 10 mass ppt, and particularly preferably 2 mass ppt to 10 mass ppt. In a case where the content of the particle-like specific metal component is within the above range, the occurrence of a defect in a semiconductor device is further reduced.

In a case where the organic solvent contains ethers, the chemical liquid may further contain alkenes. In some cases, alkenes are mixed into ethers as a byproduct produced at the time of manufacturing ethers among the organic solvents described above. Therefore, in a case where ethers are used as the organic solvent, sometimes the alkenes mixed into the ethers are incorporated into the chemical liquid.

Examples of the alkenes include ethylene, propylene, butene, pentene, heptene, octene, nonene, decene, and the like. The chemical liquid may contain only one kind of alkenes or two or more kinds of alkenes.

In a case where the chemical liquid contains the alkenes, the content of the alkenes in the chemical liquid is preferably 0.1 mass ppb to 100 mass ppb, and more preferably 0.1 mass ppb to 10 mass ppb. In a case where the content of the alkenes is within the above range, the interaction between the metal components and the alkenes can be inhibited, and the chemical liquid demonstrates better the performance.

In a case where the chemical liquid contains two or more kinds of alkenes, the aforementioned content of the alkenes means the total content of the two or more kinds of alkenes.

The content of the alkenes in the chemical liquid is measured by Gas Chromatography Mass Spectrometer (GC-MS).

The method for making the content of the alkenes in the chemical liquid fall into the above range will be described later.

### (Acid Component)

In a case where the organic solvent contains lactones, the chemical liquid may further contain at least one kind of acid component selected from an inorganic acid and an organic acid.

The acid component is used as an acid catalyst at the time of manufacturing lactones among the aforementioned organic solvents. Therefore, sometimes the acid component is mixed into the lactones. Accordingly, in a case where the lactones are used as an organic solvent, sometimes the acid component mixed into the lactones is incorporated into the chemical liquid.

Examples of the acid component include at least one kind of acid selected from an inorganic acid and an organic acid. Examples of the inorganic acid include hydrochloric acid, phosphoric acid, sulfuric acid, perchloric acid, and the like, but the inorganic acid is not limited to these. Examples of the organic acid include formic acid, methanesulfonic acid, trifluoroacetic acid, p-toluenesulfonic acid, and the like, but the organic acid is not limited to these.

In a case where the chemical liquid contains the acid component, the content of the acid component in the chemical liquid is preferably 0.1 mass ppb to 100 mass ppb, more preferably 0.1 mass ppb to 10 mass ppb, and even more preferably 0.1 mass ppb to 1 mass ppb. In a case where the content of the acid component is within the above range, the interaction between the metal components and the acid component can be inhibited, and the chemical liquid demonstrates better the performance thereof.

In a case where the chemical liquid contains two or more kinds of acid components, the aforementioned content of the acid component means the total content of the two or more kinds of acid components.

The content of the acid component in the chemical liquid is measured by a neutralization titration method. Specifically, by the neutralization titration method, the content of the acid component is measured using an automatic potentiometric titrator (tradename “MKA-610” manufactured by KYOTO ELECTRONICS MANUFACTURING CO., LTD.).

As the method for making the content of the acid component in the chemical liquid fall into the above range, for example, electrodeionization and the distillation treatment in the purification step, which will be described later, may be repeated.

### (Other Components)

The chemical liquid may contain components other than the above (hereinafter, referred to as “other components” as well) according to the use. Examples of other additives include a surfactant, an antifoaming agent, a chelating agent, and the like.

### <Organic Impurity>

It is preferable that the content of an organic impurity in the chemical liquid is small. For measuring the content of the organic impurity, a gas chromatography mass spectrometer (tradename “GCMS-2020”, manufactured by Shimadzu Corporation) is used. The measurement conditions are as described in Examples. In a case where the organic impurity is a high-molecular weight compound, the techniques such as pyrolyzer-quadrupole time-of-flight/mass spectrometry (Py-QTOF/MS), pyrolyzer-ion trapping mass spectrometry (Py-IT/MS), pyrolyzer magnetic sector field mass spectrometry (Py-Sector/MS), pyrolyzer Fourier transform ion cyclotron resonance mass spectrometry (Py-FTICR-MS), pyrolyzer quadrupole mass spectrometry (Py-Q/MS), and pyrolyzer ion trap mass time-of-flight mass spectrometry (Py-IT-TOF/MS) may be used for identifying the structure or quantifying the concentration from the decomposition product, but the measurement method is not particularly limited. For example, for Py-QTOF/MS, a device manufactured by Shimadzu Corporation or the like can be used.

### <Kit and Concentrate>

The chemical liquid can be used as a treatment liquid used at the time of manufacturing a semiconductor or as a raw material of the treatment liquid. Examples of the aspect in which the chemical liquid is used as a raw material include a kit to which other raw materials are separately added. In this case, examples of other raw materials separated added at the time of use include at least one kind of material selected from the group consisting of water, an organic solvent, a chemical liquid, and the like. Furthermore, according to the use, the chemical liquid may be used by being mixed with other compounds.

As an aspect in which the chemical liquid is used as a treatment liquid, an aspect in which the chemical liquid is used as a concentrate can be exemplified. In this case, the chemical liquid can be used by adding water, an organic solvent, and/or other compounds thereto at the time of use.

### [Manufacturing Device]

The manufacturing device as an embodiment of the present invention is a manufacturing device for manufacturing a chemical liquid, comprising a reaction portion for obtaining a reactant, which is a chemical liquid (chemical liquid for a semiconductor), by reacting a raw material, a distillation column for obtaining a purified product by distilling the reactant, and a first transfer pipeline which connects the reaction portion and the distillation column to each other so as to transfer the reactant to the distillation column from the reaction portion, in which the interior wall of the distillation column is coated with or formed of at least one kind of material (corrosion resistant material) selected from the group consisting of a fluororesin and an electropolished metal material, the metal material contains at least one kind of metal selected from the group consisting of chromium and nickel, and the total content of the chromium and the nickel is greater than 25% by mass with respect to the total mass of the metal material.

FIG. 2 is a schematic view showing the constitution of a manufacturing device 200 according to the embodiment described above.

In FIG. 2, the manufacturing device 200 includes a reaction portion 201 for obtaining a reactant, which is a chemical liquid, by reacting a raw material, and a distillation column 202 for obtaining a purified product by purifying the reactant. The interior wall of the distillation column 202 is coated with or formed of a material.

The reaction portion 201 and the distillation column 202 are connected to each other through a first transfer pipeline 203.

The manufacturing device 200 further includes a filling portion 204 for filling a container with the purified product. The distillation column 202 and the filling portion 204 are connected to each other through a second transfer pipeline 205.

In addition, the manufacturing device 200 further includes a filter portion 206 for filtering the purified product by using a filter. The filter portion 206 is disposed in a position in the middle of the second transfer pipeline 205.

Moreover, the manufacturing device 200 further includes a raw material supply portion 207 for supplying the raw material to the reaction portion 201. The reaction portion 201 and the raw material supply portion 207 are connected to each other through a third transfer pipeline 208.

### [Reaction Portion]

The reaction portion 201 has a function of obtaining a reactant, which is a chemical liquid, by reacting the raw material (if necessary, in the presence of a catalyst) supplied thereto. As the reaction portion 201, known reaction portions can be used without particular limitation.

Examples of the reaction portion 201 include an aspect including a reactor to which the raw material is supplied and in which a reaction proceeds, a stirring portion provided in the interior of the reactor, a lid portion joined to the reactor, an injection portion for injecting the raw material into the reactor, and a reactant outlet portion for taking the reactant out of the reactor. By continuously or non-continuously injecting the raw material into the reaction portion and reacting the injected raw materials (in the presence of a catalyst), a reactant which is a chemical liquid can be obtained.

If desired, the reaction portion 201 may also include a reactant isolation portion, a temperature control portion, a sensor portion including a level gauge, a manometer, and a thermometer, and the like.

In the reaction portion 201, the interior wall of the reactor is preferably coated with or formed of at least one kind of material (corrosion resistant material) selected from the group consisting of a fluororesin and an electropolished metal material. The aspect of the material is as described above.

Particularly, in view of obtaining a chemical liquid with a further reduced impurity content, the interior wall of the reactor is more preferably coated with or formed of an electropolished metal material, and even more preferably coated with electropolished stainless steel or formed of an electropolished metal material.

By the manufacturing device 200 including the aforementioned reactor, a chemical liquid with a further reduced impurity content can be obtained.

### [Distillation Column]

The interior wall of the distillation column 202 is coated with or formed of at least one kind of material (corrosion resistant material) selected from the group consisting of a fluororesin and an electropolished metal material. The aspect of the material is as described above.

An infill may be disposed in the interior of the distillation column 202 just like the distillation column 101 described above.

### [First Transfer Pipeline]

The reaction portion 201 and the distillation column 202 are connected to each other through the first transfer pipeline 203. Because the reaction portion 201 and the distillation column 202 are connected to each other through the first transfer pipeline 203, the transfer of the reactant to the distillation column 202 from the reaction portion 201 is carried out in a closed system, and impurities including metal components are inhibited from being mixed into the reactant from the environment. Accordingly, a chemical liquid with a further reduced impurity content can be obtained.

As the first transfer pipeline 203, known transfer pipelines can be used without particular limitation. As the transfer pipeline, an aspect including a pipe, a pump, a valve, and the like can be exemplified.

The interior wall of the first transfer pipeline 203 is coated with or formed of at least one kind of material (corrosion resistant material) selected from the group consisting of a fluororesin and an electropolished metal material. The aspect of the material is as described above.

Particularly, in view of obtaining a chemical liquid with a further reduced impurity content, the interior wall of the first transfer pipeline is more preferably coated with or formed of a fluororesin, and even more preferably coated with or formed of a tetrafluoroethylene-perfluoroalkyl vinyl ether copolymer.

By the manufacturing device 200 including the first transfer pipeline 203, a chemical liquid with a further reduced impurity content can be obtained.

### [Filling Portion]

The manufacturing device 200 includes the filling portion 204. The filling portion 204 has a function of filling a container with the purified product. As the filling portion 204, filling devices known to be used for filling with a liquid can be used without particular limitation.

As the filling portion 204, an aspect including a storage tank of a purified product and an injection portion which is connected to the storage tank so as to inject the purified product into a container can be exemplified. By the injection portion which is for continuously or non-continuously injecting the purified product into the storage tank and is connected to the storage tank, the purified product is injected into a container. If desired, the filling portion 204 may include a metering device of a container, a transport device of a container, and the like.

In a case where the filling portion 204 includes the storage tank, the interior wall of the storage tank is preferably coated with or formed of at least one kind of material (corrosion resistant material) selected from the group consisting of a fluororesin and an electropolished metal material. The aspect of the material is as described above.

By the manufacturing device 200 including the filling portion 204, a chemical liquid with a further reduced impurity content can be obtained.

### <Container>

As the container used in the filling portion 204, known containers can be used without particular limitation. Examples of the container include an isotainer, a drum, a pail, a bottle, and the like. As long as there is no problem with corrosion resistance and the like, any container can be used.

Among these, a container which exhibits high cleanliness with respect to the chemical liquid and hardly causes elution of impurities is preferable. Examples of the container which exhibits high cleanliness and hardly causes the elution of impurities include a “CLEAN BOTTLE” series manufactured by AICELLO CORPORATION, “PURE BOTTLE” manufactured by KODAMA PLASTICS Co., Ltd., and the like, but the container is not limited to these.

The interior wall of the container is preferably coated with or formed of a specific material which will be described later, and more preferably coated with or formed of a corrosion resistant material. The aspect of the specific material will be described later, and the aspect of the corrosion resistant material is as described above.

Particularly, in view of obtaining a chemical liquid with a further reduced impurity content, the interior wall of the container is more preferably coated with or formed of a fluororesin, and even more preferably coated with or formed of polytetrafluoroethylene.

In a case where the aforementioned container is used, the occurrence of a problem such as the elution of an oligomer of ethylene and/or a propylene can be further reduced than in a case where a container formed of other resins such as a polyethylene resin, a polypropylene resin, a polyethylene-polypropylene resin, and the like is used.

Specific examples of the container include FluoroPurePFA composite drum manufactured by Entegris, Inc., and the like. Furthermore, it is possible to use the containers described on page 4 and the like of JP1991-502677A (JP-H03-502677A), page 3 and the like of WO2004/016526A, pages 9, 16, and the like of WO99/046309A, and the like.

It is preferable to wash the interior of the container before filling. The liquid used for washing is not particularly limited, but the content of metal components in the liquid is preferably less than 0.001 mass parts per trillion (ppt). In addition, as desired, in a case where the liquid contains water and another organic solvent which is purified so as to make the metal content to fall into the aforementioned range, the aforementioned chemical liquid itself, the aforementioned chemical liquid which is diluted, or at least one kind of compound added to the aforementioned chemical liquid, a chemical liquid with a reduced metal component content can be obtained.

### [Second Transfer Pipeline]

The distillation column 202 and the filling portion 204 are connected to each other through the second transfer pipeline 205. In a case where the distillation column 202 and the filling portion 204 are connected to each other through the second transfer pipeline 205, the transfer of the purified product to the filling portion 204 from the distillation column 202 is carried out in a closed system. Accordingly, impurities including metal components are inhibited from being mixed into the purified product from the environment. As a result, a chemical liquid with a further reduced impurity content can be obtained.

The aspect of the second transfer pipeline 205 is the same as the aspect of the first transfer pipeline 203.

### [Filter Portion]

The manufacturing device 200 includes the filter portion 206. The filter portion 206 is disposed in a position in the middle of the second transfer pipeline 205 and has a function of filtering the purified product by causing the purified product to pass through a filter. As the filter portion 206, known filtering devices can be used without particular limitation.

Examples of the filter portion 206 include a filter unit including one or plural filters and a filter housing.

In FIG. 2, the filter portion 206 is disposed in a position in the middle of the second transfer pipeline 205. However, the aspect of the filter portion 206 of the manufacturing device 200 according to the embodiment described above is not limited thereto. The manufacturing device according to the embodiment described above also includes an aspect in which a plurality of filter portions 206 are disposed in series and/or in parallel in a position the middle of the second transfer pipeline 205.

### <Filter>

The material of the filter is not particularly limited, but examples thereof include a fluororesin such as polytetrafluoroethylene, a polyamide-based resin such as nylon, a polyolefin resin (including a polyolefin resin with high density and ultra-high molecular weight) such as polyethylene and polypropylene, because these materials can efficiently remove minute foreign substances such as impurities and/or aggregates contained in the chemical liquid. It is preferable that the filter is formed of at least one kind of material selected from the group consisting of nylon, polypropylene (including high-density polypropylene), polyethylene, polytetrafluoroethylene, and a tetrafluoroethylene-perfluoroalkyl vinyl ether copolymer among the above materials.

By the filter formed of the aforementioned material, foreign substances with high polarity, which readily become the cause of a residue defect and/or a particle defect, can be efficiently removed, and the content of the metal components in the chemical liquid can be efficiently reduced.

The critical surface tension of the filter is preferably equal to or higher than 70 mN/m, more preferably equal to or lower than 95 mN/m, and even more preferably equal to or higher than 75 mN/m and equal to or lower than 85 mN/m.

The value of the critical surface tension is the nominal value from manufacturers. In a case where a filter having critical surface tension within the above range is used, foreign substances with high polarity, which readily become the cause of a residue defect and/or a particle defect, can be efficiently removed, and the content of the metal components in the chemical liquid can be efficiently reduced.

The average pore size of the filter is not particularly limited, but a pore size of about 0.001 to 1.0 μm is appropriate. The average pore size is preferably about 0.002 to 0.2 μm, and more preferably about 0.005 to 0.01 μm. In a case where the average pore size is within the above range, it is possible to inhibit the clogging of the filter and to reliably remove minute foreign substances such as impurities or aggregates contained in the purified product.

From the viewpoint of reducing the content of the metal components in the chemical liquid, the average pore size of the filter is preferably equal to or smaller than 0.05 μm. For adjusting the content of the metal components in the chemical liquid, the average pore size of the filter is preferably equal to or greater than 0.005 μm and equal to or smaller than 0.05 μm, and more preferably equal to or greater than 0.01 μm and equal to or smaller than 0.02 μm. In a case where the average pore size is within the above range, the pressure necessary for filtering can be kept low, and filtering can be efficiently performed.

As the average pore size mentioned herein, the nominal values form filter manufacturers can be referred to. A commercial filter can be selected from various filters provided from, for example, Pall Corporation Japan, Advantec Toyo Kaisha, Ltd., Nihon Entegris KK (former MICRONICS JAPAN CO., LTD.), KITZ MICRO FILTER CORPORATION, or the like.

Furthermore, it is also possible to use “P-NYLON FILTER (average pore size: 0.02 μm, critical surface tension: 77 mN/m)” made of polyamide (manufactured by Pall Corporation Japan), “PE⋅CLEAN FILTER (average pore size: 0.02 μm)” made of high-density polyethylene (manufactured by Pall Corporation Japan), and “PE⋅CLEAN FILTER (average pore size: 0.01 μm)” made of high-density polyethylene (manufactured by Pall Corporation Japan).

The filter portion may include different kinds of filters (for example, a plurality of filters formed of different materials). In a case where the filter portion includes a plurality of different kinds of filters, a chemical liquid with a further reduced impurity content can be obtained. The aforementioned filtering step will be described later.

### [Raw Material Supply Portion]

The manufacturing device 200 includes the raw material supply portion 207. As the raw material supply portion 207, known raw material supply devices can be used without particular limitation, as long as the devices can continuously or non-continuously supply raw materials such as a solid, a liquid, or a gas to the reaction portion 201.

As the raw material supply portion 207, an aspect including a raw material-receiving tank, a sensor such as a level gauge, a pump, a valve for controlling the supply of the raw material, and the like can be exemplified.

The raw material supply portion 207 and the reaction portion 201 are connected to each other through the third transfer pipeline 208.

In FIG. 2, the manufacturing device 200 includes one raw material supply portion 207. However, the aspect of the manufacturing device 200 is not limited thereto, and for example, an aspect in which the manufacturing device 200 includes a plurality of parallel raw material supply portions 207 for each kind of raw material is also included in the manufacturing device 200 according to the embodiment described above.

In a case where the raw material supply portion 207 includes a raw material-receiving tank, the interior wall of the raw material-receiving tank is preferably coated with or formed of at least one kind of material selected from the group consisting of a fluororesin and an electropolished metal material. The aspect of the material is as described above.

By the manufacturing device including the raw material supply portion 207, a chemical liquid with a further reduced impurity content can be obtained.

### [Third Transfer Pipeline]

The raw material supply portion 207 and the reaction portion 201 are connected to each other through the third transfer pipeline 208. In a case where the raw material supply portion 207 and the reaction portion 201 are connected to each other through the third transfer pipeline 208, the transfer of the raw material to the reaction portion 201 from the raw material supply portion 207 is carried out in a closed system. Therefore, impurities including metal components are inhibited from being mixed into the raw material from the environment. As a result, a chemical liquid with a further reduced impurity content can be obtained.

The aspect of the third transfer pipeline 208 is the same as the aspect of the first transfer pipeline 203.

The manufacturing device 200 in FIG. 2 includes the filling portion 204, the filter portion 206, the raw material supply portion 207, the second transfer pipeline 205, and the third transfer pipeline 208. However, the manufacturing device according to an embodiment of the present invention is not limited to this aspect.

The manufacturing device according to an embodiment of the present invention may include at least the reaction portion 201, the distillation column 202, and the first transfer pipeline 203, and the interior wall of the distillation column 202 may be coated with or formed of at least a material (corrosion resistant material).

### <Raw Material>

As the raw materials used in the manufacturing device, materials known to be used for manufacturing a chemical liquid can be used without particular limitation. Among these, in view of obtaining a chemical liquid with a further reduced impurity content, a raw material having high purity is preferable. That is, it is preferable to use a raw material of a so-called high-purity grade. The purity of the raw material is not particularly limited, but is preferably equal to or higher than 99.99% and more preferably equal to or higher than 99.999%.

Due to the manufacturing process of the raw material and the like, metal components may be incorporated as impurities into the raw materials in some cases. Examples of the metal component incorporated as impurities include Na, K, Ca, Fe, Cu, Mg, Mn, Li, Al, Cr, Ni, Zn, and the like. Generally, the content of these impurities with respect to the total mass of the raw material is 0.01 to 100 mass ppm in many cases.

As the method for measuring the content of the impurities, for example, the aforementioned SP-ICP-MS method can be used.

It is preferable that the raw material is purified before being supplied for manufacturing of the chemical liquid. As the purification method, known purification methods can be used without particular limitation.

Examples of the purification method include filtering, ion exchange, distillation, and the like. In a case where distillation is performed, the aforementioned purification device may be used.

As described above, the manufacturing device 200 includes the distillation column 202. Therefore, in a case where a chemical liquid is manufactured using the manufacturing device 200, a chemical liquid with a reduced impurity content can be obtained.

### [Manufacturing Method of Chemical Liquid]

The manufacturing method of a chemical liquid according to an embodiment of the present invention is a manufacturing method of a chemical liquid, comprising a reaction step of obtaining a reactant, which is a chemical liquid, by reacting a raw material and a purification step of obtaining a purified product by distilling the reactant by using a distillation column, in which the interior wall of the distillation column is coated with or formed of at least one kind of material selected from the group consisting of a fluororesin and an electropolished metal material, the metal material contains at least one kind of metal selected from the group consisting of chromium and nickel, and the total content of the chromium and the nickel with respect to the total mass of the metal material is greater than 25% by mass.

### [Reaction Step]

The reaction step is a step of obtaining a reactant, which is a chemical liquid, by reacting a raw material.

The reactant is not particularly limited, and examples thereof include the aspect described above as a chemical liquid. That is, examples of the reaction step include a step of synthesizing the compound (A) for obtaining a chemical liquid containing the compound (A).

As the method for obtaining the reactant, known methods can be used without particular limitation. Examples thereof include a method of obtaining a reactant by reacting one or plural raw materials in the presence of a catalyst.

More specifically, examples thereof include a step of obtaining butyl acetate by reacting acetic acid and n-butanol in the presence of sulfuric acid, a step of obtaining 1-hexanol by reacting ethylene, oxygen, and water in the presence of Al(C₂H₅)₃, a step of obtaining 4-methyl-2-pentanol by reacting cis-4-methyl-2-pentene in the presence of Diisopinocamphenylborane (Ipc2BH), a step of obtaining propylene glycol 1-monomethyl ether 2-acetate (PGMEA) by reacting propylene oxide, methanol, and acetic acid with each other in the presence of sulfuric acid, a step of obtaining isopropyl alcohol (IPA) by reacting acetone with hydrogen in the presence of copper oxide-zinc oxide-aluminum oxide, a step of obtaining ethyl lactate by reacting lactic acid with ethanol, and the like.

### [Purification Step]

The purification step is a step of obtaining a purified product by distilling the reactant. The purification step is performed using the distillation column described above. The method for obtaining a purified product by distilling the reactant by using the distillation column has already been described above.

According to the manufacturing method described above, the interior wall of the distillation column is coated with or formed of a material. Therefore, a chemical liquid with a reduced impurity content can be obtained.

It is preferable that the manufacturing method of a chemical liquid according to an embodiment of the present invention further comprises a filtering step of filtering the purified product by using a filter after the purification step.

### [Filtering Step]

The filtering step is preferably a step of passing the purified product through a filter. The method for passing the purified product through a filter is not particularly limited, and examples thereof include a method of disposing a filter unit including a filter and a filter housing in the middle of a transfer pipeline transferring the purified product and passing the purified product through the filter unit with or without applying pressure thereto.

The aspect of the filter to be used is as described above.

For the filtering step, an aspect may be adopted in which the purified product is filtered plural times by using filters formed of different materials, filters of different average pore sizes (hereinafter, referred to as “pore size” as well), and the like. Particularly, an aspect is more preferable in which the purified product is filtered plural times by using filters formed of different materials.

In this case, the filtering performed using a first filter may be carried out once or twice or more times. In a case where different filters are combined and filtering is performed twice or more times, it is preferable that the pore size of the filter for the first filtering is the same as or larger than the pore size of the filters for the second filtering and the following filtering. Furthermore, filters having different pore sizes within the range of the average pore size described above may be combined. Regarding the pore size mentioned herein, the nominal values from filter manufacturers can be referred to. A commercial filter can be selected from various filters provided from, for example, Pall Corporation Japan, Advantec Toyo Kaisha, Ltd., Nihon Entegris KK (former MICRONICS JAPAN CO., LTD.), KITZ MICRO FILTER CORPORATION, or the like.

A second filter may be formed of a material different from that of the first filter.

A suitable pore size of the second filter is about 0.01 to 1.0 μm, and a preferable pore size of the second filter is about 0.1 to 0.5 μm. By making the pore size fall into this range, in a case where the chemical liquid contains component particles, it is possible to remove foreign substances mixed into the chemical liquid while leaving the component particles.

In a case where a filter having a pore size smaller than that of the first filter is used as the second filter, a ratio of the pore size of the second filter to the pore size of the first filter (pore size of the second filter/pore size of the first filter) is preferably 0.01 to 0.99, more preferably 0.1 to 0.9, and even more preferably 0.3 to 0.9.

For example, the filtering in the first filter may be performed using a mixed liquid containing some of the components of the chemical liquid, the mixed liquid may be mixed with other components so as to prepare the chemical liquid, and then the second filtering may be performed.

It is preferable that the filter to be used is treated before filtering a chemical liquid. The liquid used for the treatment is not particularly limited, but it is preferable that the metal content in the liquid is less than 0.001 mass parts per trillion (ppt). As such a liquid, for example, a liquid obtained by purifying ultrapure water, water, and/or an organic solvent for manufacturing a semiconductor so as to make the metal content fall into the aforementioned range, the chemical liquid itself, a liquid obtained by diluting the chemical liquid, or a liquid containing the compound added to the chemical liquid is preferable.

The temperature at which the filtering step is performed is preferably equal to or lower than room temperature (25° C.), more preferably equal to or lower than 23° C., and even more preferably equal to or lower than 20° C. Furthermore, the temperature is preferably equal to or higher than 0° C., more preferably equal to or higher than 5° C., and even more preferably equal to or higher than 10° C.

In the filtering step, particle-like foreign substance and/or impurities can be removed. In a case where the filtering step is performed at the temperature described above, because the content of particle-like foreign substances and impurities dissolved in the chemical liquid is reduced, the foreign substances and/or the impurities are more efficiently removed by the filtering step.

Particularly, in a case where the chemical liquid contains metal components containing an extremely trace amount of element selected from the group consisting of Fe, Ni, Pt, Pd, and Al, it is preferable to performing filtering at the aforementioned temperature. Presumably, in a case where the chemical liquid contains an extremely trace amount of element, which is desirable in the present application, selected from the group consisting of Fe, Ni, Pt, Pd, and Al, most of the metal components may be present in the state of particle-like colloid, although the mechanism is unclear. In a case where the filtering is performed at the aforementioned temperature, some of the metal components floating in the colloidal state are aggregated. Presumably, as a result, the aggregated metal components may be efficiently removed by the filtering, or the content of the metal components can be easily adjusted and become a desired content.

It is preferable that the filtering step is performed using the aforementioned manufacturing device. Particularly, it is preferable that the filtering step is performed using a manufacturing device including the filter portion 206 for filtering the purified product by using a filter, in which the filter portion 206 is disposed in a position in the middle of the second transfer pipeline 205. In a case where the aforementioned manufacturing device is used, the filtering step can be performed in a closed system, and it is possible to inhibit impurities including metal components from being mixed into the purified product from the environment. Accordingly, a chemical liquid with a further reduced impurity content can be obtained.

### [Filling Step]

The aforementioned manufacturing method of a chemical liquid may further comprise a filling step of filling a container with the purified product. As the filling method, known filling methods can be used without particular limitation. The aspect of the container which can be used in the filling step is as described above.

It is preferable that the filling step is performed using a manufacturing device including the filling portion 204. In a case where the filling step is performed using the manufacturing device including the filling portion 204, because the filling portion 204 is connected to the distillation column 202 or the filter portion 206 through the second transfer pipeline 205, the purification step or the filtering step and the transfer of the purified product in the filling step can be performed in a closed system. Therefore, it is possible to inhibit impurities including metal components from being mixed into the purified product from the environment. Accordingly, a chemical liquid with a further reduced impurity content can be obtained.

Examples of suitable aspects of the manufacturing method of a chemical liquid include a method of performing each of the aforementioned steps by using the manufacturing device 200. In this case, a part, which is wet with a liquid, of each portion in the manufacturing device 200 is preferably coated with or formed of a material.

Specifically, it is preferable that the interior wall of each of the distillation column 202 and the reaction portion 201 is coated with or formed of an electropolished metal material.

It is preferable that the interior wall of each of the first and second transfer pipelines (203 and 205) is coated with or formed of a fluororesin.

According to the above aspect, each step is performed in a closed system. Therefore, impurities including metal components are inhibited from being mixed into the purified product from the environment, and metal components are hardly eluted from each portion of the manufacturing device. As a result, a chemical liquid with a further reduced impurity content can be obtained.

If desired, the manufacturing method of a chemical liquid according to the embodiment described above may further include a raw material supply step, a destaticizing step, and the like, in addition to the steps described above.

### [Raw Material Supply Step]

The raw material supply step is a step of supplying a raw material used in the reaction step. The method of supplying a raw material used in the reaction step is not particularly limited, and examples thereof include a method of supplying a raw material to the reaction portion 201 by using the raw material supply portion 207, and the like.

In a case where the raw material supply step is performed using the manufacturing device 200 including the raw material supply portion 207, the transfer of the raw material to the reaction portion 201 from the raw material supply portion 207 is performed in a closed system. Therefore, impurities including metal components are inhibited from being mixed into the raw material from the environment. Accordingly, a chemical liquid with a further reduced impurity content can be obtained.

At this time, the interior wall of the receiving tank of the raw material supply portion 207 and the storage tank of the filling portion 204 is preferably coated with or formed of a material.

The interior wall of the third transfer pipeline 208 is preferably coated with or formed of a fluororesin.

### [Destaticizing Step]

The destaticizing step is a step of removing electricity of at least one kind of component (hereinafter, referred to as “purified product or the like”) selected from the group consisting of a raw material, a reactant, and a purified product so as to reduce the charge potential of the purified product or the like.

As the destaticizing method, known destaticizing methods can be used without particular limitation. Examples of the destaticizing method include a method of bringing the purified product or the like into contact with a conductive material, and the like.

The purified product or the like is brought into contact with a conductive material preferably for 0.001 to 60 seconds, more preferably for 0.001 to 1 second, and even more preferably for 0.01 to 0.1 seconds. Examples of the conductive material include stainless steel, gold, platinum, diamond, glassy carbon, and the like.

Examples of the method of bringing the purified product or the like into contact with a conductive material include a method of disposing a grounded mesh formed of a conductive material in the interior of a pipeline and passing the purified product or the like through the mesh, and the like.

The manufacturing method of a chemical liquid preferably includes the destaticizing step before at least one kind of step selected from the group consisting of the raw material supply step, the reaction step, the purification step, the filtering step, and the filling step.

For example, the destaticizing step is preferably performed before the purified product or the like is injected into the receiving tank which may be included in the raw material supply portion 207, the reactor which may be included in the reaction portion 201, the distillation column 202, a container to be filled, and the like. In a case where the destaticizing step is performed as described above, it is possible to inhibit impurities derived from a container and the like from being mixed into the purified product or the like.

It is preferable that all of the preparation of a chemical liquid, the opening of a storage container, the washing of an empty container, analysis, and the like are performed in a clean room. It is preferable that the clean room meets the 14644-1 clean room standard. The clean room preferably meets any of International Organization for Standardization (ISO) class 1, ISO class 2, ISO class 3, and ISO class 4, more preferably meets ISO class 1 or ISO class 2, and even more preferably meets ISO class 1.

By the aforementioned manufacturing method of a chemical liquid, a chemical liquid with a reduced impurity content can be obtained. Specifically, it is possible to obtain a chemical liquid in which the content of metal components as impurities is reduced such that the concentration of the compound (A) becomes 99.9% to 99.9999999% by mass. The aspect of the compound (A) is as described above.

In a case where the aforementioned chemical liquid is used as a raw material of a treatment liquid for a semiconductor, as another raw material, at least one kind of material selected from the group consisting of water, an organic solvent, and a chemical liquid can be exemplified.

In a case where the chemical liquid is used as a raw material of the treatment liquid for a semiconductor, it is preferable that the chemical liquid and another raw material are purified before these are mixed together. The aspect of the purification method is as described above as the purification method of the raw material.

Furthermore, in a case where the chemical liquid is used as a raw material of the treatment liquid for a semiconductor, it is preferable that the treatment liquid for a semiconductor is purified before the treatment liquid is mixed with another raw material. The aspect of the purification method is as described above.

It is even more preferable that the manufacturing method of a chemical liquid further includes a step of purifying the raw material.

It is preferable that the aforementioned chemical liquid is used as at least one kind of agent selected from the group consisting of a pre-wet liquid, a developer, and a rinsing liquid for manufacturing a semiconductor.

In an aspect, the chemical liquid is preferably used as a developer, a rinsing liquid, or a pre-wet liquid for forming a pattern in the semiconductor manufacturing process.

The pattern forming method include a resist film forming step of forming an actinic ray-sensitive or radiation-sensitive film (hereinafter, referred to as “resist film” as well) by coating a substrate with an actinic ray-sensitive or radiation-sensitive composition (hereinafter, referred to as “resist composition” as well), an exposure step of exposing the resist film, and a treatment step of treating the substrate coated with the resist composition or the exposed resist film with the aforementioned chemical liquid.

In the pattern forming method, the chemical liquid may be used as any of the developer, the rinsing liquid, and the pre-wet liquid. The chemical liquid is preferably used as any two agents among the developer, the rinsing liquid, and the pre-wet liquid, and more preferably used as the developer, the rinsing liquid, and the pre-wet liquid.

### [Container]

The container according to an embodiment of the present invention is a container for storing a chemical liquid (chemical liquid for a semiconductor), in which the interior wall of the container is coated with or formed of at least one kind of material (specific material) selected from the group consisting of a polyolefin resin, a fluororesin, a metal material, and an electropolished metal material, the metal material contains at least one kind of metal selected from the group consisting of chromium and nickel, and the total content of the chromium and the nickel with respect to the total mass of the metal material is greater than 25% by mass.

The interior wall of the container is coated with or formed of at least one kind of material selected from the group consisting of a polyolefin resin, a fluororesin, a metal material, and an electropolished metal material. Accordingly, even in a case where the chemical liquid is stored in the container for a long period of time, the impurity content hardly increases.

As the container, a container is preferable which can inhibit the content of a particle-like metal (particle-like metal component, referred to as “metal particles” as well) in a chemical liquid filled into the container from increasing over time, and can maintain the content of the particle-like metal in the chemical liquid within a range of 0.01% to 100% by mass even after the chemical liquid is stored in the container for a long period of time.

In an aspect, the container includes a storage portion in which the chemical liquid is stored and a seal portion sealing the storage portion.

In an aspect of the container, a proportion of void portions occupying the storage portion in which the chemical liquid is stored (hereinafter, referred to as “void volume” as well) is preferably 50% to 0.01% by volume. In a case where the upper limit of the void volume in the storage portion is equal to or lower than 50% by volume, it is possible to reduce the likelihood that impurities in a gas occupying the void portions may be mixed into the chemical liquid. In an aspect, the void volume in the storage portion is more preferably 20% to 0.01% by volume, and even more preferably 10% to 1% by volume.

In an aspect of the container, the void portions of the storage portion in which the chemical liquid is stored are preferably filled with a high-purity gas containing few particles. As such a gas, for example, a gas in which the number of particles having a diameter equal to or greater than 0.5 μm is equal to or smaller than 10 per 1 L is preferable, and a gas in which the number of particles having a diameter equal to or greater than 0.5 μm is 1 per 1 L is more preferable.

### [Material (Specific Material)]

The material (specific material) is at least one kind of material selected from the group consisting of a polyolefin resin, a fluororesin, a metal material, and an electropolished metal material.

### <Metal Material>

The metal material contains at least one kind of metal selected from the group consisting of chromium and nickel, in which the total content of the chromium and the nickel with respect to the total mass of the metal material is greater than 25% by mass. The aspect of the metal material is as described above.

As the material, an electropolished metal material is preferable. The aspect of the electropolished metal material is as described above as the metal material having undergone electropolishing. The metal material may have undergone buffing. The aspect of the buffing is as described above.

In a case where the interior wall of the container is formed of a metal material having undergone electropolishing, and the metal material contains chromium and iron, the mass ratio (Cr/Fe) of Cr atoms contained in the surface of the interior wall of the container to Fe atoms contained in the surface of the interior wall of the container is not particularly limited, but is preferably equal to or higher than 0.60, more preferably equal to or higher than 0.80, even more preferably equal to or higher than 1.0, particularly preferably equal to or higher than 1.5, and most preferably higher than 1.5. Furthermore, Cr/Fe is preferably equal to or lower than 3.5, more preferably equal to or lower than 3.2, even more preferably equal to or lower than 3.0, and particularly preferably less than 2.5.

In a case where Cr/Fe is 0.80 to 3.0, even though the chemical liquid is stored for a predetermined period of time, the impurity content hardly increases.

In an aspect, it is preferable that at least a portion of the interior wall of the storage portion of the container contacting the chemical liquid is formed of a material containing at least one kind of material selected from the group consisting of stainless steel, HASTELLOY, INCONEL, and MONEL. Herein, “at least a portion” means that a lining, a lining layer, and a laminate layer used in the interior wall of the storage portion, a sealing material used in the joining portion, a lid, an observation window, and the like may be formed of other materials.

### <Fluororesin>

The aspect of the fluororesin is as described above.

### <Polyolefin Resin>

As the polyolefin resin, known polyolefin resins can be used without particular limitation. Among these, polyethylene or polypropylene is preferable. The polyolefin resin may be any of a high-density polyolefin resin and an ultrahigh molecular-weight polyolefin resin.

In an aspect, at least a portion of the interior wall of the storage portion of the container contacting the chemical liquid is preferably formed of a material containing at least one kind of compound selected from the group consisting of polyethylene, polypropylene, polytetrafluoroethylene, and perfluoroalkoxyalkane. Herein, “at least a portion” means that a lining, a lining layer, and a laminate layer used in the interior wall of the storage portion, a sealing material used in the joining portion, a lid, an observation window, and the like may be formed of other materials.

In a case where the interior wall of the container is coated with at least one kind of resin material selected from the group consisting of a polyolefin resin and a fluororesin, and the resin material forms a coating layer, the water contact angle on the outermost surface of the coating layer is not particularly limited, but is preferably equal to or greater than 90°. The upper limit of the water contact angle is not particularly limited, but is preferably equal to or smaller than 150° in general, more preferably equal to or smaller than 130°, and even more preferably less than 120°.

In a case where the interior wall of the container is formed of a resin material, the water contact angle on the outermost surface of the interior wall of the container is not particularly limited, but is preferably equal to or greater than 90°. The upper limit of the water contact angle is not particularly limited, but is preferably equal to or smaller than 150° in general, more preferably equal to or smaller than 130°, and even more preferably less than 120°.

In a case where the water contact angle on the outermost surface of the interior wall or the coating layer of the container is equal to or greater than 90°, even though the chemical liquid is stored for a predetermined period of time, the impurity content hardly increases.

It is preferable that the chemical liquid is stored in the aforementioned container. The chemical liquid is as described above. More specifically, examples thereof include the chemical liquids described in the aspects 1 to 4 of the chemical liquid. Furthermore, the following chemical liquid may also be used.

### (Aspect A of Chemical Liquid)

In an aspect, the chemical liquid preferably stored in the aforementioned container may be a chemical liquid containing metal components containing at least one kind of element selected from the group consisting of Al, Ca, Cr, Co, Cu, Fe, Pb, Li, Mg, Mn, Ni, K, Ag, Na, Ti, and Zn, in which the content of metal particles, which contain the above element, among the metal components is equal to or smaller than 100 mass ppt with respect to the total mass of the chemical liquid.

In a case where the chemical liquid, in which the content of the metal particles in the chemical liquid is controlled and become equal to or smaller than 100 mass ppt with respect to the total mass of the chemical liquid, is used as a treatment liquid for a semiconductor, the occurrence of a defect becomes harder. The content of the metal particles in the chemical liquid is more preferably equal to or smaller than 50 mass ppt with respect to the total mass of the chemical liquid, and even more preferably equal to or smaller than 10 mass ppt with respect to the total mass of the chemical liquid, because then the occurrence of a defect becomes harder in a case where the chemical liquid is used as a treatment liquid for a semiconductor.

### (Aspect B of Chemical Liquid)

In another aspect, the chemical liquid preferably stored in the aforementioned container may be a chemical liquid containing metal components containing at least one kind of element selected from the group consisting of Na, K, Ca, Fe, Cr, Ti and Ni, in which the content of metal particles, which contain the above element, among the metal components is equal to or smaller than 50 mass ppt with respect to the total mass of the chemical liquid.

The content of the metal particles in the chemical liquid is more preferably equal to or smaller than 10 mass ppt with respect to the total mass of the chemical liquid, because then the occurrence of a defect becomes harder in a case where the chemical liquid is used as a treatment liquid for a semiconductor.

Typically, the metal particles containing at least one kind of element selected from the group consisting of Na, K, Ca, Fe, Cr, Ti and Ni represent metal particles containing Na, metal particles containing K, metal particles containing Ca, metal particles containing Fe, metal particles containing Cr, metal particles containing Ti, metal particles containing Ni, and the like.

In a case where the chemical liquid contains one kind of particles described above, the content of the metal particles with respect to the total mass of the chemical liquid is equal to or smaller than 50 mass ppt, and preferably equal to or smaller than 10 mass ppt. In a case where the chemical liquid contains a plurality of kinds of metal particles described above, the content of each kind of particles with respect to the total mass of the chemical liquid is equal to or smaller than 50 mass ppt, and preferably equal to or smaller than 10 mass ppt.

### (Aspect C of Chemical Liquid)

In a still another aspect, the chemical liquid preferably stored in the aforementioned container may be a chemical liquid in which the chemical liquid contains metal components containing Fe, and the content of metal particles, which contain Fe, among the metal components is equal to or smaller than 10 mass ppt with respect to the total mass of the chemical liquid.

The chemical liquid is preferably used for manufacturing a semiconductor. Specifically, in a manufacturing process of a semiconductor device including a lithography step, an etching step, an ion implantation step, a peeling step, and the like, the chemical liquid is used for treating an organic substance after each step is finished or before the next step is started. Specifically, the chemical liquid is suitably used as a pre-wet liquid, a developer, a rinsing liquid, a peeling liquid, and the like.

The chemical liquid can also be suitably used for other uses in addition to the manufacturing of a semiconductor. The chemical liquid can be used as a developer or a rinsing liquid of polyimide, a resist for a sensor, a resist for a lens, and the like.

Furthermore, the chemical liquid can also be used for washing. The chemical liquid can be suitably used for washing containers, piping, substrates (for example, a wafer and glass), and the like. Specifically, the chemical liquid is suitably used as a washing liquid, a remover, a peeling liquid, and the like.

Specifically, for the purpose of removing inorganic metal ions on a silicon substrate, the chemical liquid is mixed with hydrochloric and suitably used for removing metal ions from the surface of a silicon substrate by a chemical liquid treatment called standard clean (SC)-2. Alternatively, for the purpose of removing particles on a silicon substrate, the chemical liquid is mixed with ammonia and suitably used for removing silicon particles from the surface of a silicon substrate by a chemical liquid treatment called standard clean (SC)-1. In addition, for the purpose of removing a resist on a substrate, the chemical liquid is mixed with sulfuric acid and suitably used for removing the resist from the substrate surface by a chemical liquid treatment called Sulfuric Acid Hydrogen Peroxide Mixture (SPM). Among these, as described above, in a manufacturing process of a semiconductor device including a lithography step, an etching step, and an ion implantation step, the chemical liquid is used for treating an organic substance after each step is finished or before the next step is started. For example, the chemical liquid is used as a developer, a rinsing liquid, an etching liquid, a washing liquid, a peeling liquid, and the like.

From the viewpoint of inhibiting the increase of particle-like metal having a particle size equal to or greater than 30 nm, which is relatively large particle size, in a case where the chemical liquid is stored for a long period of time, provided that an interaction radius in the space of Hansen solubility parameters (HSP) derived from the material of a filter used for filtering is R0 and that the radius of a sphere as the Hansen space derived from the liquid contained in the chemical liquid is Ra, the chemical liquid to be stored in the aforementioned container is preferably a liquid which forms a combination with a filter so as to satisfy a relational expression (Ra/RO)≤1 and is filtered through a filter material satisfying the relational expression. Ra/RO is preferably equal to or smaller than 0.98, and more preferably equal to or smaller than 0.95. The lower limit of Ra/R0 is preferably equal to or smaller than 0.5, more preferably equal to or greater than 0.6, and even more preferably 0.7. In a case where Ra/R0 is within the above range, the formation of particle-like metal having a relative large particle size and the growth of particle-like metal is inhibited at the time of long-term storage, the metal components contained in the container of the present invention are hardly eluted into the chemical liquid, and as a result, the increase of particle-like metal having a particle size equal to or greater than 30 nm is inhibited, although the mechanism is unclear.

The combination of the filter and the liquid is not particularly limited, but examples thereof include those described in US2016/0089622.

### [Manufacturing Method of Container]

The manufacturing method of the aforementioned container is not particularly limited, and the container can be manufactured by known methods. For example, by a method of bonding a fluororesin lining to the interior wall of a container formed of a metal, a resin, or the like, a method of coating the interior wall of a distillation column formed of a metal, a resin, or the like with a composition containing a fluororesin or a polyolefin resin so as to form a coat, and the like, a container whose interior wall is coated with a material can be manufactured.

Furthermore, for example, by a method of electropolishing the interior wall of a container formed of a metal material in which the total content of chromium and nickel with respect to the total mass of the metal material is greater than 25% by mass, and the like, a container whose interior wall is formed of an electropolished metal material can be manufactured.

### [Manufacturing Method of Chemical Liquid]

The manufacturing method of a chemical liquid according to an embodiment of the present invention is a manufacturing method of a chemical liquid in which the aforementioned container is filled with the purified product in the filling step.

The manufacturing method of a chemical liquid may further comprise a filling step of filling a container with the purified product. As the filling method, known filling methods can be used without particular limitation. The aspect of the container which can be used in the filling step is as described above.

The aspects of steps other than the above steps are as described above.

The manufacturing method of a chemical liquid according to an embodiment of the present invention is a manufacturing method of a chemical liquid, which further comprises a step of washing the interior wall of the container by using a washing liquid before the filling step described above, in which the contact angle of the washing liquid with respect to the interior wall is 10° to 120°.

As the method of washing the interior wall of the container by using a washing liquid, known methods can be used without particular limitation.

Examples of the method of washing the interior wall of the container by using a washing liquid include Example 1 and Example 2 described below.

### Example 1

A container having an internal volume of 20 L is filled with 5 L of a washing liquid and then closed. Thereafter, vibration stirring is performed for 1 minute such that the washing liquid is evenly spread on the entire surface of the interior portion of the container that is wet with the liquid, and then the lid is opened so as to discharge the washing liquid. Subsequently, the container is thoroughly rinsed by being washed out 3 times with ultrapure water and then dried. The number of times of washing performed using the washing liquid and the washing time are determined according to the required cleanliness and/or the number of times of rinsing performed after the washing by using the ultrapure water and the rinsing time are determined as necessary.

### Example 2

The opening portion of a container is caused to face downward, and through a jetting nozzle or the like, a washing liquid is jetted from the opening portion to the inner surface of the container such that the container is washed. In order to make it possible to wash the entirety of the inner surface of the container, a method of using a diffusion nozzle, a method of disposing a plurality of nozzles, a method of washing the container in a state of moving the container and/or the washing nozzle, and the like are appropriately performed. The washing time is determined according to the required cleanliness.

### [Washing Liquid]

The contact angle of the washing liquid, which is used for washing the interior wall, with respect to the interior wall of the container is 10° to 120°.

The contact angle is an indicator involved in the wettability of the surface of a certain substance with respect to a certain liquid. The contact angle is represented by an angle θ formed between a tangent to the edge of a liquid (washing liquid) attached to a substance (interior wall of the storage portion) and a surface of the substance. Accordingly, the larger the contact angle θ is, the easier it is for the substance to repel the liquid, and the lower the wettability with respect to the liquid. Inversely, the smaller the contact angle θ is, the more difficult it is for the substance to repel the liquid, and the higher the wettability with respect to the liquid. The size of the contact angle θ depends on the intensity of the surface energy. The lower the surface energy, the larger the contact angle θ. In the present specification, the contact angle is a value measured by the θ/2 method.

In a case where the contact angle of the washing liquid with respect to the interior wall is equal to or greater than 10°, the washing liquid hardly remains in the container after the washing is finished, and it is possible to inhibit the washing liquid and/or contaminants contained in the washing liquid from being mixed as impurities into the chemical liquid to be filled into the container after washing.

Furthermore, in a case where the contact angle of the washing liquid with respect to the interior wall is equal to or smaller than 120°, it is possible to increase the removal rate of contaminants remaining in tiny voids of the storage portion and the like.

In addition, the manufacturing method of a chemical liquid according to an embodiment of the present invention is a manufacturing method, in which the chemical liquid is a chemical liquid containing at least one kind of component selected from the group consisting of water and an organic solvent, and the washing liquid is at least one kind of liquid selected from the group consisting of a chemical liquid, an organic solvent, water, and a mixture of these.

Generally, in manufacturing a high-purity chemical liquid, the washing liquid itself can become an impurity. However, according to the manufacturing method described above, before the filling step, the container is washed with at least one kind of liquid selected from the group consisting of a chemical liquid, an organic solvent, water, and a mixture of these. Therefore, it is possible to inhibit the washing liquid from becoming a cause of the occurrence of impurities. In other words, in a case where a washing liquid containing the same components as those in the chemical liquid is used, the occurrence of impurities can be further inhibited.

Specific examples of the washing liquid include ultrapure water, isopropyl alcohol, and the like. As the ultrapure water or the isopropyl alcohol used as a washing liquid in the present invention, those of a grade, in which inorganic ions such as sulfate ions, chloride ions, or nitrate ions or Fe, Cu, and Zn as target metals are reduced, are preferably used. Alternatively, further purified ultrapure water or isopropyl alcohol is preferably used. The purification method is not particularly limited, but either or both of the purification using a filtration membrane and/or an ion-exchange membrane and/or the purification by distillation are preferable.

The aspects of the chemical liquid and the organic solvent which can be used as a washing liquid are as described above.

The chemical liquid storage container according to an embodiment of the present invention is a chemical liquid storage container comprising a container and a chemical liquid stored in the container.

By the chemical liquid storage container, even in a case where the chemical liquid is stored for a predetermined period of time, impurities (for example, metal particles and/or coarse particles) in the chemical liquid hardly increase.

The aspect of the container is as described above. Furthermore, the aspect of the chemical liquid is as described above as “Aspect 1 of chemical liquid” to “Aspect 4 of chemical liquid” in the present specification.

The aforementioned chemical liquid may contain metal components containing at least one kind of element selected from the group consisting of Al, Ca, Cr, Co, Cu, Fe, Pb, Li, Mg, Mn, Ni, K, Ag, Na, Ti, and Zn, in which the content of metal particles, which contain the above element, among the metal components may be equal to or smaller than 100 mass ppt with respect to the total mass of the chemical liquid.

The chemical liquid has already been described above as Aspect A of chemical liquid.

Furthermore, the aforementioned chemical liquid may contain metal components containing at least one kind of element selected from the group consisting of Na, K, Ca, Fe, Cr, Ti, and Ni, in which the content of metal particles, which contain the above element, among the metal components may be equal to or smaller than 50 mass ppt with respect to the total mass of the chemical liquid. The chemical liquid has already been described above as Aspect B of chemical liquid.

In addition, the aforementioned chemical liquid may contain metal components containing Fe, in which the content of metal particles, which contain Fe, among the metal components may be equal to or smaller than 10 mass ppt with respect to the total mass of the chemical liquid. The chemical liquid has already been described above as Aspect C of chemical liquid.

### Examples

The present invention will be more specifically described based on examples. The materials, the amount and proportion of the materials used, the details of treatments, the procedure of treatments, and the like shown in the following examples can be appropriately modified as long as the gist of the present invention is maintained. Accordingly, the scope of the present invention is not limited to the following examples.

### [Preparation of Chemical Liquid]

Hereinafter, a chemical liquid preparation method will be described.

### [Purification of Raw Material and the Like]

Each raw material and each catalyst used in each of the examples described below were those of grades having a purity equal to or higher than 99% by mass, and purified in advance by distillation, ion exchange, filtering, and the like.

The ultrapure water used for preparing each chemical liquid was purified by the method described in JP2007-254168A. Thereafter, whether the content of each of the elements including Na, Ca, and Fe is less than 10 mass ppt with respect to the total mass of each chemical liquid was checked by the measurement by the SP-ICP-MS method which will be described later, and then the ultrapure water was used.

The preparation, filling, storage, and analysis of the chemical liquid in each of the examples and the comparative examples were performed in a clean room of a level that meets a standard equal to or lower than ISO class 2. Furthermore, the container used in each of the examples and the comparative examples was washed with the chemical liquid of each of the examples or the comparative examples before being used. In order to improve the accuracy of measurement, at the time of measuring the content of metal components and measuring the content of water, in a case where the content of metal components or the content of water was found to be equal to or smaller than a detection limit by general measurement, the content was measured by concentrating the chemical liquid by a factor of 0.01 in terms of the volume, and the content was calculated by converting the concentration into the concentration of the chemical liquid not yet being concentrated.

### [Preparation of Manufacturing Device]

The chemical liquid of each of the examples and comparative examples was prepared using a manufacturing device including a reactor, a distillation column, and a filter portion including 1 to 4 stages.

The reactor, the distillation column, the filter portion, and the container were connected to each other through a transfer pipeline.

The interior wall of each portion (the reactor, the distillation column, the transfer pipeline, or the like) was formed of the material shown in Table 1. Each of the abbreviations in Table 1 represents the following material.

In a case where PTFE or PFA was used, a coat of the corresponding material was formed on the interior wall surface of each portion. In a case where SUS316EP or Buffed SUS316 was used, the interior wall of each portion was formed of the corresponding material.
Buffed SUS316+EP: material obtained by buffing SUS316 (stainless steel; Ni content of 10% by mass, Cr content of 16% by mass) with #400 grains and then performing electropolishing
Buffed SUS316: material obtained by buffing SUS316 with #400 grains cloth
SUS316EP: material obtained by electropolishing SUS316
PTFE: polytetrafluoroethylene
PFA: tetrafluoroethylene-perfluoroalkyl vinyl ether copolymer

Table 1 shows the type of each of the filters of stages 1 to 4 in the filter portion and the average pore size thereof (value listed in a catalog). Each of the abbreviations in Table 1 represents the following filter.
PP: filter made of polypropylene (manufactured by 3M Japan Ltd, NanoSHIELD)
HDPE: filter made of high-density polyethylene (manufactured by Pall Corporation Japan, PE CLEAN)
Nylon: filter made of 66 nylon (manufactured by Pall Corporation Japan, ULTIPLEAT)
PTFE: filter made of polytetrafluoroethylene (manufactured by Nihon Entegris KK, TORRENTO)

The electropolishing of stainless steel was performed under the following condition. The current density, interelectrode distance, and/or electropolishing time were adjusted such that Cr/Fe in each member became the value described in Table 1.

### <Electropolishing Condition>

Electropolishing solution: “S-CLEAN EP” manufactured by SASAKI CHEMICAL CO., LTD.

Temperature: 50° C. to 60° C.

Time: 2 to 10 minutes

Current density: 10 to 20 A/dm³

Interelectrode distance: 5 to 50 cm

### [Preparation of Container]

The chemical liquid of each of the examples and the comparative examples was prepared by the method described below and then filled into a container. Table 1 shows the material of the container used. Each of the abbreviations in Table 1 represents the following container.
PTFE: (container made of polytetrafluoroethylene)
SUS316EP: (container made of electropolished SUS316)
Buffed SUS316+EP: material obtained by buffing SUS316 (stainless steel; Ni content of 10% by mass, Cr content of 16% by mass) with #400 grains and then performing electropolishing
Buffed SUS316: material obtained by buffing SUS316 with #400 grains

### Example 1

### (Step 1)

Acetic acid and n-butanol were reacted with each other in the reactor in the presence of sulfuric acid as a catalyst. Then, the obtained reactant was introduced into the distillation column and reacted in a state where water generated as a by-product in the form of an azeotropic mixture of butyl acetate/n-butanol/water was being removed to the outside from the system through an outlet on top of the distillation column, thereby obtaining a crude liquid 1b containing butyl acetate (hereinafter, referred to as “butyl acetate crude liquid”).

### (Step 2)

The sulfuric acid fraction in the butyl acetate crude liquid 1b obtained by the step 1 was neutralized using an alkali. Thereafter, the crude liquid was washed with water, and then moisture was removed, thereby obtaining a butyl acetate crude liquid 1c.

### (Step 3)

The butyl acetate crude liquid 1c obtained by the step 2 was neutralized and washed with water, and most of the water and the sulfuric acid was separated using a decanter. Then, a butyl acetate crude liquid 1d containing butyl acetate, n-butanol, water, sulfuric acid, and a trace of by-product was supplied to the distillation column so as to remove low boiling point substances such as n-butanol as an impurity and water. Thereafter, distillation was repeated plural times, thereby obtaining a chemical liquid.

As the method of repeating distillation plural times, a method was used in which the purified product obtained after distillation was taken out at the position in the middle of the aforementioned transfer pipeline and returned to the transfer pipeline in front of the distillation column.

Thereafter, the chemical liquid was filtered through the filter portion including a plurality of filters described below in which a portion wet with the liquid is made of PFA and which are disposed in a position in the middle of the transfer pipeline, and filled into a container made of polytetrafluoroethylene. The container made of polytetrafluoroethylene was pre-washed with the chemical liquid of Example 1 before being filled.

### Filter Constitution

Stage 1: made of polytetrafluoroethylene, average pore size of 20 nm

Stage 2: made of 66 nylon, average pore size of 10 nm

Stage 3: made of polytetrafluoroethylene, average pore size of 10 nm

Stage 4: made of 66 nylon, average pore size of 5 nm

### Examples 2 to 7, 10 to 14, and 20 to 33 and Comparative Examples 1 to 3

By using a manufacturing device in which the interior wall of each portion was formed of the material described in Table 1 and which included filters of the material and the average pore size described in Table 1, a chemical liquid was manufactured by the same method as the method described in Example 1 (Steps 1 to 3). Before the chemical liquid was filled into a container whose interior wall was formed of the material described in Table 1, the container was washed with the washing liquid described in Table 1, thereby obtaining chemical liquid storage containers of Examples 2 to 7, Examples 10 to 14, and Comparative Examples 1 to 3. “Pre-washing” in the column of washing liquid in Table 1 means that the chemical liquid according to the example or the comparative example was used as a washing liquid.

The chemical liquid of Example 11 was repeatedly distilled until the moisture content became about 1/10 of the moisture content in the chemical liquid described in Example 1.

In Table 1, “Cr/Fe” described for the material of the interior wall of each portion of the manufacturing device of the chemical liquid represents a mass ratio of Cr atoms contained in the surface to Fe atoms contained in the surface. For determining Cr/Fe, the presence of each of the element species was confirmed by qualitative analysis by using an X-ray Photoelectron Spectroscopy (XPS) device “Quantum 2000” manufactured by ULVAC-PHI, INCORPORATED. The concentration of each of the confirmed elements was evaluated by quantitative measurement, and the ratio Cr/Fe was calculated. Cr/Fe was measured using an X-ray having a beam diameter of 200 μm and an Al-kα X-ray source with pass energy of 140.0 eV and a step size of 0.125 eV under the Ar etching condition.

In Table 1, “C.A.” described for the material of the interior wall of each portion of the manufacturing device of the chemical liquid represents a water contact angle (unit: “°”) on the outermost surface. The water contact angle was measured using a full automatic contact angle meter DMo-701 manufactured by Kyowa Interface Science Co., LTD under the condition of room temperature (23° C.).

### Example 8

### (Step 1)

By using acetone and hydrogen, an acetone reduction reaction was performed according to a known method in the presence of copper oxide-zinc oxide-aluminum oxide as a catalyst. In this reaction, a heating treatment was performed for 4 hours at 100° C., thereby obtaining an IPA-containing crude liquid (hereinafter, referred to as “IPA crude liquid”) 2a.

### (Step 2)

The IPA crude liquid 2a contained unreacted acetone, a substituted isomer as an impurity, and a catalyst. For the purpose of purifying the IPA crude liquid 2a, the WA crude liquid 2a was introduced into the distillation column. Then, distillation was repeated plural times, thereby obtaining a chemical liquid.

Then, the chemical liquid was filtered through the filter portion including a plurality of filters described below in which a portion wet with the liquid was made of PFA and which were disposed in a position in the middle of the transfer pipeline.

### Filter Constitution

Stage 1: made of polytetrafluoroethylene, average pore size of 10 nm

Stage 2: made of high-density polyethylene, average pore size of 10 nm

Then, the chemical liquid was filled into a container made of polytetrafluoroethylene.

### Example 9 and Comparative Examples 4 and 5

By using a manufacturing device in which the interior wall of each portion was formed of the material described in Table 1 and which included filters of the material and the average pore size described in Table 1, a chemical liquid was manufactured by the same method as the method described in Example 8 (Steps 1 and 2). Before the chemical liquid was filled into a container whose interior wall was formed of the material described in Table 1, the container was washed with the washing liquid described in Table 1, thereby obtaining chemical liquid storage containers of Example 9 and Comparative Examples 4 and 5.

### Examples 15 to 19

According to a known method, crude liquids containing cyclohexanone, propylene glycol 1-monomethyl ether 2-acetate (PGMEA), ethyl lactate, isoamyl acetate (IAA), and methyl isobutyl carbinol (MIBC) respectively were manufactured. Then, by using a manufacturing device in which the interior wall of each portion was formed of the material described in Table 1 and which included filters of the material and the average pore size described in Table 1, a chemical liquid was manufactured. Before the chemical liquid was filled into a container whose interior wall was formed of the material described in Table 1, the container was washed with the washing liquid described in Table 1, thereby obtaining chemical liquid storage containers of Examples 15 to 19.

The solvent content in each of the chemical liquids of Examples 1 to 33 and Comparative Examples 1 to 5 was measured by the Karl Fischer moisture measurement method using a Karl Fischer moisture meter (coulometric titration method) MKC-710M and a method of measuring the amount of a totally evaporated residue. The results are shown in Table 1. The solvent content represents the amount (% by mass) of butyl acetate or IPA with respect to the total mass of the chemical liquid.

### [Evaluation: Measurement of Content of Metal Components]

For measuring the content of metal components, 1,000 mL of the chemical liquid was put into a container made of synthetic quartz and subjected to ashing by being heated using a muffle furnace such that the chemical liquid could be kept boiling. The sample obtained after the ashing was dissolved in ultrapure water, thereby preparing a sample solution. The sample solution was measured using Inductively Coupled Plasma Mass Spectrometry (ICP-MS). The measurement results were evaluated based on the following standards, and summarized in Table 1. The unit of each value is mass parts per trillion (ppt). For practical use, the evaluation result that meets D or a higher standard is preferable.

A: The content of metal components was less than 50 mass ppt.

B: The content of metal components was equal to or greater than 50 mass ppt and less than 100 mass ppt.

C: The content of metal components was equal to or greater than 100 mass ppt and less than 500 mass ppt.

D: The content of metal components was equal to or greater than 500 mass ppt and less than 10,000 mass ppt.

E: The content of metal components was equal to or greater than 10,000 mass ppt.

The manufacturing device used for manufacturing a chemical liquid and the evaluation result of the chemical liquid manufactured using the manufacturing device are described in Table 1 which is divided into Table 1-1 to Table 1-4.

For example, in Example 1, butyl acetate was used as the compound (A), the interior wall of the reactor was formed of the buffed SUS316+EP (Cr/Fe equaled 2.0), the interior wall of the distillation column was formed of the buffed SUS316+Ep (Cr/Fe equaled 2.0), the interior wall of the transfer pipeline was formed of PFA (C.A. was 100°), the chemical liquid was prepared using a manufacturing device including a stage 1 filter made of PTFE and having an average pore size of 20 nm, a stage 2 filter made of nylon and having an average pore size of 10 nm, a stage 3 filter made of PTFE and having an average pore size of 20 nm, and a stage 4 filter made of nylon and having an average pore size of 5 nm, and a container whose interior wall was made of PTFE (C.A. was 115°) was filled with the chemical liquid after being pre-washed with the chemical liquid. In the obtained chemical liquid, the content of the solvent (compound (A)) was 99.9999999% by mass, the content of a metal component containing Na was 7.0 mass ppt, the content of a metal component containing K was 3.0 mass ppt, the content of a metal component containing Ca was 3.0 mass ppt, the content of a metal component containing Fe was less than 1.0 mass ppt, the content of a metal component containing Ni was less than 1.0 mass ppt, the content of a metal component containing Cr was less than 1.0 mass ppt, the content of a metal component containing Ti was less than 2.0 mass ppt, the total content of these metal components was 15 mass ppt, and the evaluation result was “A”. Other examples in Table 1 can be interpreted in this way

TABLE 1-1

| | | Manufacturing device | | | | | |
|---|---|---|---|---|---|---|---|
| | | Interior wall of each portion | | | | | |
| | | Reactor | | | Distillation column | | |
| | Compound (A) | Material | Cr/Fe | C.A. | Material | Cr/Fe | C.A. |
| Example 1 | Butyl acetate | Buffed SUS316 + EP | 2.0 | | Buffed SUS316 + EP | 2.0 | |
| Example 2 | Butyl acetate | Buffed SUS316 + EP | 2.0 | | Buffed SUS316 + EP | 2.0 | |
| Example 3 | Butyl acetate | Buffed SUS316 + EP | 2.0 | | Buffed SUS316 + EP | 2.0 | |
| Example 4 | Butyl acetate | Buffed SUS316 + EP | 2.0 | | PTFE | | 115 |
| Example 5 | Butyl acetate | Buffed SUS316 + EP | 2.0 | | Buffed SUS316 + EP | 2.0 | |
| Example 6 | Butyl acetate | Buffed SUS316 | 0.60 | | Buffed SUS316 + EP | 2.0 | |
| Example 7 | Butyl acetate | Buffed SUS316 + EP | 2.0 | | Buffed SUS316 + EP | 2.0 | |
| Example 8 | IPA | Buffed SUS316 + EP | 2.0 | | Buffed SUS316 + EP | 2.0 | |
| Example 9 | IPA | Buffed SUS316 + EP | 2.0 | | Buffed SUS316 + EP | 2.0 | |
| Example 10 | Butyl acetate | Buffed SUS316 + EP | 2.0 | | Buffed SUS316 + EP | 2.0 | |
| Example 11 | Butyl acetate | Buffed SUS316 + EP | 2.0 | | Buffed SUS316 + EP | 2.0 | |
| Example 12 | Butyl acetate | Buffed SUS316 + EP | 2.0 | | Buffed SUS316 + EP | 2.0 | |
| Example 13 | Butyl acetate | Buffed SUS316 + EP | 2.0 | | Buffed SUS316 + EP | 2.0 | |
| Example 14 | Butyl acetate | Buffed SUS316 + EP | 2.0 | | Buffed SUS316 + EP | 2.0 | |
| Example 15 | Cyclohexanone | Buffed SUS316 + EP | 2.0 | | Buffed SUS316 + EP | 2.0 | |
| Example 16 | PGMEA | Buffed SUS316 + EP | 2.0 | | Buffed SUS316 + EP | 2.0 | |
| Example 17 | Ethyl lactate | Buffed SUS316 + EP | 2.0 | | Buffed SUS316 + EP | 2.0 | |
| Example 18 | IAA | Buffed SUS316 + EP | 2.0 | | Buffed SUS316 + EP | 2.0 | |
| Example 19 | MIBC | Buffed SUS316 + EP | 2.0 | | Buffed SUS316 + EP | 2.0 | |
| Example 20 | Butyl acetate | Buffed SUS316 + EP | 1.5 | | Buffed SUS316 + EP | 2.0 | |
| Example 21 | Butyl acetate | Buffed SUS316 + EP | 2.0 | | Buffed SUS316 + EP | 0.70 | |
| Example 22 | Butyl acetate | Buffed SUS316 + EP | 2.0 | | Buffed SUS316 + EP | 2.0 | |
| Example 23 | Butyl acetate | Buffed SUS316 + EP | 3.2 | | Buffed SUS316 + EP | 2.0 | |
| Example 24 | Butyl acetate | Buffed SUS316 + EP | 2.0 | | Buffed SUS316 + EP | 3.2 | |
| Example 25 | Butyl acetate | Buffed SUS316 + EP | 2.0 | | Buffed SUS316 + EP | 2.0 | |
| Example 26 | Butyl acetate | Buffed SUS316 + EP | 3.0 | | Buffed SUS316 + EP | 2.0 | |
| Example 27 | Butyl acetate | Buffed SUS316 + EP | 2.0 | | Buffed SUS316 + EP | 3.0 | |
| Example 28 | Butyl acetate | Buffed SUS316 + EP | 2.0 | | Buffed SUS316 + EP | 2.0 | |
| Example 29 | Butyl acetate | Buffed SUS316 + EP | 1.0 | | Buffed SUS316 + EP | 2.0 | |
| Example 30 | Butyl acetate | Buffed SUS316 + EP | 2.0 | | Buffed SUS316 + EP | 1.0 | |
| Example 31 | Butyl acetate | Buffed SUS316 + EP | 2.0 | | Buffed SUS316 + EP | 2.0 | |
| Example 33 | Butyl acetate | Buffed SUS316 + EP | 2.0 | | PTFE | | 115 |
| Example 34 | Butyl acetate | Buffed SUS316 + EP | 2.0 | | PTFE | | 115 |
| Example 35 | Butyl acetate | Buffed SUS316 + EP | 2.0 | | PFA | | 100 |
| Example 36 | Butyl acetate | Buffed SUS316 + EP | 2.0 | | PFA | | 100 |
| Comparative | Butyl acetate | Buffed SUS316 + EP | 2.0 | | Buffed SUS316 | 2.0 | |
| Example 1 | | | | | | | |
| Comparative | Butyl acetate | Buffed SUS316 | 2.0 | | Buffed SUS316 | 2.0 | |
| Example 2 | | | | | | | |
| Comparative | Butyl acetate | Buffed SUS316 | 2.0 | | Buffed SUS316 | 2.0 | |
| Example 3 | | | | | | | |
| Comparative | IPA | Buffed SUS316 + EP | 2.0 | | Buffed SUS316 | 2.0 | |
| Example 4 | | | | | | | |
| Comparative | IPA | Buffed SUS316 | 2.0 | | Buffed SUS316 | 2.0 | |
| Example 5 | | | | | | | |
| Example 38 | Butyl acetate | Buffed SUS316 + EP | 2.0 | | Buffed SUS316 + EP | 2.0 | |
| Example 39 | Butyl acetate | Buffed SUS316 + EP | 2.0 | | Buffed SUS316 + EP | 2.0 | |
| Example 40 | Butyl acetate | PTFE | | 115 | Buffed SUS316 + EP | 2.0 | |

TABLE 1-2

| | Manufacturing device | | | | | | |
|---|---|---|---|---|---|---|---|
| | | | | Filter portion | | | |
| | Interior wall of each portion | | | Stage 1 | | Stage 2 | |
| | Transfer pipeline | | | | Average pore | | Average pore |
| | Material | Cr/Fe | C.A. | Material | size | Material | size |
| Example 1 | PFA | | 100 | PTFE | 20 nm | Nylon | 10 nm |
| Example 2 | PFA | | 100 | Nylon | 10 nm | PTFE | 20 nm |
| Example 3 | PFA | | 100 | PTFE | 20 nm | Nylon | 10 nm |
| Example 4 | PFA | | 100 | PTFE | 20 nm | Nylon | 10 nm |
| Example 5 | PFA | | 100 | PTFE | 20 nm | PTFE | 10 nm |
| Example 6 | PFA | | 100 | Nylon | 10 nm | PTFE | 20 nm |
| Example 7 | Buffed SUS316 | 0.60 | | Nylon | 10 nm | PTFE | 20 nm |
| Example 8 | PFA | | 100 | PTFE | 20 nm | HDPE | 5 nm |
| Example 9 | PFA | | 100 | PP | 10 nm | PTFE | 20 nm |
| Example 10 | PFA | | 100 | Nylon | 10 nm | PTFE | 20 nm |
| Example 11 | PFA | | 100 | Nylon | 10 nm | PTFE | 20 nm |
| Example 12 | Buffed SUS316 + EP | 2.0 | | Nylon | 10 nm | PTFE | 20 nm |
| Example 13 | Buffed SUS316 + EP | 2.0 | | Nylon | 10 nm | PTFE | 20 nm |
| Example 14 | Buffed SUS316 + EP | 2.0 | | Nylon | 10 nm | PTFE | 20 nm |
| Example 15 | Buffed SUS316 + EP | 2.0 | | Nylon | 10 nm | PTFE | 20 nm |
| Example 16 | Buffed SUS316 + EP | 2.0 | | Nylon | 10 nm | PTFE | 20 nm |
| Example 17 | Buffed SUS316 + EP | 2.0 | | Nylon | 10 nm | PTFE | 20 nm |
| Example 18 | Buffed SUS316 + EP | 2.0 | | Nylon | 10 nm | PTFE | 20 nm |
| Example 19 | Buffed SUS316 + EP | 2.0 | | Nylon | 10 nm | PTFE | 20 nm |
| Example 20 | Buffed SUS316 + EP | 2.0 | | Nylon | 10 nm | PTFE | 20 nm |
| Example 21 | Buffed SUS316 + EP | 2.0 | | Nylon | 10 nm | PTFE | 20 nm |
| Example 22 | Buffed SUS316 + EP | 1.5 | | Nylon | 10 nm | PTFE | 20 nm |
| Example 23 | Buffed SUS316 + EP | 2.0 | | Nylon | 10 nm | PTFE | 20 nm |
| Example 24 | Buffed SUS316 + EP | 2.0 | | Nylon | 10 nm | PTFE | 20 nm |
| Example 25 | Buffed SUS316 + EP | 3.2 | | Nylon | 10 nm | PTFE | 20 nm |
| Example 26 | Buffed SUS316 + EP | 2.0 | | Nylon | 10 nm | PTFE | 20 nm |
| Example 27 | Buffed SUS316 + EP | 2.0 | | Nylon | 10 nm | PTFE | 20 nm |
| Example 28 | Buffed SUS316 + EP | 3.0 | | Nylon | 10 nm | PTFE | 20 nm |
| Example 29 | Buffed SUS316 + EP | 2.0 | | Nylon | 10 nm | PTFE | 20 nm |
| Example 30 | Buffed SUS316 + EP | 2.0 | | Nylon | 10 nm | PTFE | 20 nm |
| Example 31 | Buffed SUS316 + EP | 1.0 | | Nylon | 10 nm | PTFE | 20 nm |
| Example 33 | PFA | | 100 | PTFE | 20 nm | Nylon | 10 nm |
| Example 34 | PFA | | 90 | PTFE | 20 nm | Nylon | 10 nm |
| Example 35 | PFA | | 100 | PTFE | 20 nm | Nylon | 10 nm |
| Example 36 | PFA | | 100 | PTFE | 20 nm | Nylon | 10 nm |
| Comparative | PFA | | 100 | Nylon | 10 nm | PTFE | 20 nm |
| Example 1 | | | | | | | |
| Comparative | Buffed SUS316 | 0.60 | | Nylon | 10 nm | PTFE | 20 nm |
| Example 2 | | | | | | | |
| Comparative | Buffed SUS316 | 0.60 | | PTFE | 20 nm | | |
| Example 3 | | | | | | | |
| Comparative | PFA | | 100 | PTFE | 20 nm | HDPE | 5 nm |
| Example 4 | | | | | | | |
| Comparative | Buffed SUS316 | 0.60 | | PTFE | 20 nm | HDPE | 5 nm |
| Example 5 | | | | | | | |
| Example 38 | PFA | | 100 | PTFE | 20 nm | Nylon | 10 nm |
| Example 39 | PFA | | 100 | PTFE | 20 nm | Nylon | 10 nm |
| Example 40 | PFA | | 100 | PTFE | 20 nm | Nylon | 10 nm |

TABLE 1-3

| | Manufacturing device | | | | | | | |
|---|---|---|---|---|---|---|---|---|
| | Filter portion | | | | Container | | | |
| | Stage 3 | | Stage 4 | | | | | |
| | | Average pore | | Average pore | Interior wall | | | |
| | Material | size | Material | size | Material | Cr/Fe | C.A. | Washing |
| Example 1 | PTFE | 20 nm | Nylon | 5 nm | PTFE | | 115 | Pre-washing |
| Example 2 | | | | | PTFE | | 115 | Pre-washing |
| Example 3 | | | | | PTFE | | 115 | Pre-washing |
| Example 4 | PTFE | 20 nm | Nylon | 5 nm | PTFE | | 115 | Pre-washing |
| Example 5 | | | | | PTFE | | 115 | Pre-washing |
| Example 6 | | | | | PTFE | | 115 | Pre-washing |
| Example 7 | | | | | PTFE | | 115 | Pre-washing |
| Example 8 | | | | | PTFE | | 115 | Pre-washing |
| Example 9 | | | | | PTFE | | 115 | Pre-washing |
| Example 10 | | | | | PTFE | | 115 | Ultrapure |
| | | | | | | | | water |
| Example 11 | | | | | PTFE | | 115 | Pre-washing |
| Example 12 | | | | | Buffed SUS316 + EP | 2 | | Pre-washing |
| Example 13 | | | | | Buffed SUS316 | 0.6 | | Pre-washing |
| Example 14 | | | | | SUS316EP | 0.4 | | Pre-washing |
| Example 15 | | | | | PTFE | | 115 | Pre-washing |
| Example 16 | | | | | PTFE | | 115 | Pre-washing |
| Example 17 | | | | | PTFE | | 115 | Pre-washing |
| Example 18 | | | | | PTFE | | 115 | Pre-washing |
| Example 19 | | | | | PTFE | | 115 | Pre-washing |
| Example 20 | | | | | PTFE | | 115 | Pre-washing |
| Example 21 | | | | | PTFE | | 115 | Pre-washing |
| Example 22 | | | | | PTFE | | 115 | Pre-washing |
| Example 23 | | | | | PTFE | | 115 | Pre-washing |
| Example 24 | | | | | PTFE | | 115 | Pre-washing |
| Example 25 | | | | | PTFE | | 115 | Pre-washing |
| Example 26 | | | | | PTFE | | 115 | Pre-washing |
| Example 27 | | | | | PTFE | | 115 | Pre-washing |
| Example 28 | | | | | PTFE | | 115 | Pre-washing |
| Example 29 | | | | | PTFE | | 115 | Pre-washing |
| Example 30 | | | | | PTFE | | 115 | Pre-washing |
| Example 31 | | | | | PTFE | | 115 | Pre-washing |
| Example 33 | PTFE | 20 nm | Nylon | 5 nm | PTFE | | 115 | Pre-washing |
| Example 34 | PTFE | 20 nm | Nylon | 5 nm | PTFE | | 115 | Pre-washing |
| Example 35 | PTFE | 20 nm | Nylon | 5 nm | PTFE | | 115 | Pre-washing |
| Example 36 | PTFE | 20 nm | Nylon | 5 nm | PTFE | | 115 | Pre-washing |
| Comparative | | | | | PTFE | | 115 | Pre-washing |
| Example 1 | | | | | | | | |
| Comparative | | | | | PTFE | | 115 | Pre-washing |
| Example 2 | | | | | | | | |
| Comparative | | | | | PTFE | | 115 | Pre-washing |
| Example 3 | | | | | | | | |
| Comparative | | | | | PTFE | | 115 | Pre-washing |
| Example 4 | | | | | | | | |
| Comparative | | | | | Buffed SUS316 | 0.6 | | Pre-washing |
| Example 5 | | | | | | | | |
| Example 38 | | | | | Buffed SUS316 + EP | 3.2 | | Pre-washing |
| Example 39 | | | | | Buffed SUS316 + EP | 1.5 | | Pre-washing |
| Example 40 | PTFE | 20 nm | Nylon | 5 nm | PTFE | | 115 | Pre-washing |

TABLE 1-4

| | Chemical liquid | | | | | | | | | |
|---|---|---|---|---|---|---|---|---|---|---|
| | Solvent content | Content of metal component (mass ppt) | | | | | | | | |
| | (% by mass) | Na | K | Ca | Fe | Ni | Cr | Ti | Total | Evaluation |
| Example 1 | 99.9999999 | 7.0 | 3.0 | 3.0 | <1 | <1 | <1 | 2.0 | 15 | A |
| Example 2 | 99.9999999 | 15 | 12 | 7.0 | 2.0 | 1 | <1 | 4.3 | 41 | A |
| Example 3 | 99.9999999 | 14 | 12 | 8.0 | <1 | <1 | <1 | 4.0 | 38 | A |
| Example 4 | 99.9999999 | 8.0 | 2.0 | 5.0 | <1 | <1 | <1 | 2.3 | 17 | A |
| Example 5 | 99.9999999 | 21 | 11 | 8.0 | 9.0 | 7.0 | 1.0 | 6.0 | 63 | B |
| Example 6 | 99.9999999 | 125 | 118 | 95 | 89 | 147 | 132 | 112 | 818 | D |
| Example 7 | 99.9999999 | 58 | 21 | 11 | 79 | 46 | 12 | 17 | 244 | C |
| Example 8 | 99.9999999 | 11 | 4.0 | 5.0 | 2.0 | 1.0 | <1 | 3.1 | 26 | A |
| Example 9 | 99.9999999 | 14 | 7.0 | 8.0 | 8.0 | 5.0 | 2.0 | 4.0 | 48 | A |
| Example 10 | 99.7 | 39 | 21 | 11 | 29 | 18 | 3.0 | 11 | 132 | C |
| Example 11 | 99.99999999 | 14 | 11 | 3.0 | 19 | 12 | 2.0 | 4.0 | 65 | B |
| Example 12 | 99.9999999 | 11 | 6.0 | 2.0 | 9.0 | 7.0 | 2.0 | 3.1 | 40 | A |
| Example 13 | 99.9999999 | 63 | 24 | 9.0 | 89 | 67 | 23 | 18 | 293 | C |
| Example 14 | 99.9999999 | 21 | 14 | 5.0 | 22 | 17 | 8.0 | 6.0 | 93 | B |
| Example 15 | 99.9999999 | 9.0 | 1.0 | 4.0 | <1 | <1 | <1 | 2.6 | 17 | A |
| Example 16 | 99.9999999 | 7.0 | 2.0 | 5.0 | <1 | <1 | <1 | 2.0 | 16 | A |
| Example 17 | 99.9999999 | 8.0 | 1.0 | 6.0 | <1 | <1 | <1 | 2.3 | 17 | A |
| Example 18 | 99.9999999 | 6.0 | 2.0 | 5.0 | <1 | <1 | <1 | 1.7 | 15 | A |
| Example 19 | 99.9999999 | 1.5 | 3.8 | 3.2 | 2.5 | 4.0 | 8.4 | 2.2 | 26 | A |
| Example 20 | 99.9999999 | 16 | 14 | 16 | 25 | 13 | 5.0 | 4.6 | 94 | B |
| Example 21 | 99.9999999 | 254 | 321 | 178 | 335 | 485 | 123 | 324 | 2,020 | D |
| Example 22 | 99.9999999 | 19 | 11 | 5.0 | 22 | 13 | 7.0 | 6.0 | 93 | B |
| Example 23 | 99.9999999 | 12 | 23 | 45 | 32 | 14 | 25 | 75 | 226 | C |
| Example 24 | 99.9999999 | 55 | 23 | 12 | 74 | 51 | 13 | 16 | 244 | C |
| Example 25 | 99.9999999 | 20 | 14 | 16 | 21 | 14 | 47 | 36 | 168 | C |
| Example 26 | 99.9999999 | 13 | 11 | 12 | 17 | 16 | 5.0 | 3.7 | 78 | B |
| Example 27 | 99.9999999 | 14 | 10 | 11 | 17 | 15 | 4.0 | 4.0 | 75 | B |
| Example 28 | 99.9999999 | 18 | 13 | 15 | 24 | 17 | 6.0 | 5.1 | 98 | B |
| Example 29 | 99.9999999 | 17 | 21 | 15 | 24 | 35 | 7.0 | 4.9 | 124 | C |
| Example 30 | 99.9999999 | 25 | 23 | 11 | 13 | 14 | 15 | 7.1 | 108 | C |
| Example 31 | 99.9999999 | 23 | 16 | 15 | 30 | 19 | 9.0 | 6.6 | 119 | C |
| Example 33 | 99.9999999 | 11 | 3.0 | 4.0 | <1 | <1 | <1 | 3.1 | 21 | A |
| Example 34 | 99.9999999 | 6.2 | 2.3 | 8.6 | <1 | <1 | <1 | 1.8 | 19 | A |
| Example 35 | 99.9999999 | 12 | 14 | 11 | <1 | <1 | <1 | 3.4 | 40 | A |
| Example 36 | 99.9999999 | 6.2 | 9.3 | 5.6 | 8.0 | 9.2 | 6.5 | 1.2 | 46 | A |
| Comparative | 99.9999999 | 2,130 | 402 | 253 | 19,021 | 1,231 | 432 | 609 | 24,078 | E |
| Example 1 | | | | | | | | | | |
| Comparative | 99.9999999 | 1,429 | 556 | 231 | 18,830 | 12,201 | 201 | 408 | 33,856 | E |
| Example 2 | | | | | | | | | | |
| Comparative | 99.9999999 | 1,723 | 689 | 219 | 18,723 | 9,899 | 316 | 492 | 32,061 | E |
| Example 3 | | | | | | | | | | |
| Comparative | 99.9999999 | 1,523 | 453 | 254 | 16,116 | 17,230 | 230 | 435 | 36,241 | E |
| Example 4 | | | | | | | | | | |
| Comparative | 99.9999999 | 3,880 | 1,180 | 320 | 36,870 | 21,800 | 2,200 | 1,109 | 67,359 | E |
| Example 5 | | | | | | | | | | |
| Example 38 | 99.9999999 | 5.9 | 3.2 | 1.2 | 39 | 24 | 8.4 | 1.7 | 83 | B |
| Example 39 | 99.9999999 | 64 | 25 | 10 | 85 | 55 | 9.0 | 18 | 267 | C |
| Example 40 | 99.9999999 | 7.0 | 3.0 | 3.0 | <1 | <1 | <1 | 2.0 | 15 | A |

In Table 1, the oblique line means that a filter was not used, and “<1” means that the measured value was less than 1.0.

As is evident from the results described in Table 1, the chemical liquids of Examples 1 to 40 manufactured by a predetermined manufacturing method brought about desired effects. In contrast, the chemical liquids of Comparative Examples 1 to 5 did not bring about desired effects.

By the manufacturing method of a chemical liquid of Example 2 in which the purified product was filtered plural times by using different kinds of filters in the filtering step, the impurity content in the obtained chemical liquid was further reduced compared to the impurity content in the chemical liquid obtained by the manufacturing method of a chemical liquid of Example 5.

In the chemical liquid of Example 2 manufactured using the manufacturing device in which Cr/Fe in the interior wall of the reactor was equal to or higher than 0.8, the content of metal components was smaller than that in the chemical liquid of Example 6.

In the chemical liquid of Example 2 manufactured using the manufacturing device in which Cr/Fe in the interior wall of the transfer pipeline was equal to or higher than 0.8, the content of metal components was smaller than that in the chemical liquid of Example 7.

In the chemical liquid of Example 2 manufactured using the manufacturing device in which Cr/Fe in the interior wall of the distillation column was equal to or higher than 0.8, the content of metal components was smaller than that in the chemical liquid of Example 21.

In the chemical liquid of Example 2 manufactured using the manufacturing device in which Cr/Fe in the interior wall of the reactor was equal to or lower than 3.0, the content of metal components was smaller than that in the chemical liquid of Example 23.

In the chemical liquid of Example 2 manufactured using the manufacturing device in which Cr/Fe in the interior wall of the distillation column was equal to or lower than 3.0, the content of metal components was smaller than that in the chemical liquid of Example 24.

In the chemical liquid of Example 2 manufactured using the manufacturing device in which Cr/Fe in the interior wall of the transfer pipeline was equal to or lower than 3.0, the content of metal components was smaller than that in the chemical liquid of Example 25.

In the chemical liquid of Example 2 manufactured using the manufacturing device in which the interior wall of the reactor was formed of electropolished stainless steel, the content of metal components was smaller than that in the chemical liquid of Example 6.

In the chemical liquid of Example 2 manufactured using the manufacturing device in which the interior wall of the transfer pipeline was formed of PFA, the content of metal components was smaller than that in the chemical liquid of Example 7.

In the chemical liquid of Example 2 filtered through filters formed of different materials, the content of metal components was smaller than that in the chemical liquid of Example 5.

In the chemical liquid of Example 2 in which the interior wall of the container was washed with the chemical liquid, the content of metal components was smaller than that in the chemical liquid of Example 10.

### [Storage Test]

The container described in Table 1 was filled with each of the chemical liquids of Examples 12, 13, and 14, closed, and stored for 60 days in a thermostat at 50° C. Then, the content of metal components and the content of metal particles were measured. The content of metal components was measured by the same method as that described above, and the content of metal particles was measured by a method using SP-ICP-MS described below.

The measurement results were evaluated based on the following standards, and summarized in Table 2. The unit of each value is mass parts per trillion (ppt). For practical use, the evaluation result that meets D or a higher standard is preferable.

“A”: The content of metal particles measured after the storage for 60 days in the thermostat at 50° C. was less than 10 mass ppt with respect to the total mass of the chemical liquid.

“B”: The content of metal particles measured after the storage for 60 days in the thermostat at 50° C. was equal to or greater than 10 mass ppt and less than 50 mass ppt with respect to the total mass of the chemical liquid.

“C”: The content of metal particles measured after the storage for 60 days in the thermostat at 50° C. was equal to or greater than 50 mass ppt and less than 100 mass ppt with respect to the total mass of the chemical liquid.

### (Preparation of Standard Substance)

Ultrapure water was measured and put into a clean glass container, metal particles to be measured having a median diameter of 50 nm were added thereto such that the concentration thereof became 10,000 particles/ml, and then the mixture was treated for 30 minutes in an ultrasonic cleaning machine. A dispersion obtained in this way was used as a standard substance for measuring transport efficiency.

### (SP-ICP-MS Device Used)

Manufacturer: PerkinElmer Inc.

Model: NexION350S

### (Measurement Condition of SP-ICP-MS)

For SP-ICP-MS, by using a coaxial nebulizer made of PFA, a cyclonic spray chamber made of quartz, and a torch injector made of quartz having an inner diameter of 1 mm, the liquid to be measured was aspirated at a rate of about 0.2 mL/min. The amount of oxygen added was 0.1 L/min, plasma power was set to be 1,600 W, and cell purging was performed using an ammonia gas. The analysis was performed at a time resolution of 50 μs.

The content of metal particles and the content of metal atoms were measured using the attached analysis software described below provided from the manufacturer.
Content of metal particles: Syngistix nanoapplication module dedicated to nanoparticle analysis “SP-ICP-MS”
Content of metal atoms: Syngistix for ICP-MS software

TABLE 2-1

| | Example 12 | | | | Example 13 | | | |
|---|---|---|---|---|---|---|---|---|
| | Content of metal components | | Content of metal particles | | Content of metal components | | Content of metal particles | |
| | Immediately after | | Immediately after | | Immediately after | | Immediately after | |
| | preparation | After storage | preparation | After storage | preparation | After storage | preparation | After storage |
| | (mass ppt) | (mass ppt) | (mass ppt) | (mass ppt) | (mass ppt) | (mass ppt) | (mass ppt) | (mass ppt) |
| Na | 11 | 12 | 1 | 1 | 63 | 91 | 8 | 11 |
| K | 6 | 7 | <1 | 2 | 24 | 71 | 3 | 9 |
| Ca | 2 | 3 | <1 | 1 | 9 | 26 | <1 | 3 |
| Fe | 9 | 32 | <1 | 2 | 89 | 380 | 11 | 35 |
| Ni | 7 | 23 | <1 | 2 | 67 | 301 | 8 | 29 |
| Cr | 2 | 4 | <1 | 1 | 23 | 83 | 2 | 8 |
| Total | 37 | 81 | 1 | 9 | 275 | 952 | 32 | 95 |

| | | |
|---|---|---|
| Evaluation | A | C |

TABLE 2-2

| | Example 14 | | | |
|---|---|---|---|---|
| | Content of metal components | | Content of metal particles | |
| | Immediately after | After | Immediately after | After |
| | preparation | storage | preparation | storage |
| | (mass ppt) | (mass ppt) | (mass ppt) | (mass ppt) |
| Na | 21 | 33 | 2 | 3 |
| K | 14 | 26 | 2 | 3 |
| Ca | 5 | 16 | <1 | 2 |
| Fe | 22 | 132 | 3 | 14 |
| Ni | 17 | 128 | 2 | 14 |
| Cr | 8 | 38 | <1 | 4 |
| Total | 87 | 373 | 9 | 40 |

| | |
|---|---|
| Evaluation | B |

### EXPLANATION OF REFERENCES

100: purification device

101: distillation column

102: supply port

103: outlet

104: reboiler

105: outlet

106: condenser

107: transfer pipeline

201: reaction portion

202: distillation column

203: first transfer pipeline

204: filling portion

205: second transfer pipeline

206: filter portion

207: raw material supply portion

208: third transfer pipeline

1. A purification device comprising:
a distillation column for purifying a chemical liquid,
wherein an interior wall of the distillation column is coated with or formed of at least one kind of material selected from the group consisting of a fluororesin and an electropolished metal material,
the metal material contains at least one kind of metal selected from the group consisting of chromium and nickel, and
a total content of the chromium and the nickel is greater than 25% by mass with respect to a total mass of the metal material.

2. The purification device according to claim 1,
wherein in a case where the interior wall of the distillation column is coated with the fluororesin, and the fluororesin forms a coating layer, a water contact angle on an outermost surface of the coating layer is equal to or greater than 90°, or
in a case where the interior wall of the distillation column is formed of the fluororesin, a water contact angle on an outermost surface of the interior wall of the distillation column is equal to or greater than 90°.

3. The purification device according to claim 1,
wherein in a case where the interior wall of the distillation column is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the interior wall of the distillation column is formed of the electropolished metal material, and the metal material contains chromium and iron, a mass ratio of chromium atoms contained in a surface of the interior wall of the distillation column to iron atoms contained in the surface of the interior wall of the distillation column is 0.80 to 3.0.

4. The purification device according to claim 1,
wherein an infill is disposed in the interior of the distillation column, and
the infill is coated with or formed of at least one kind of material selected from the group consisting of a fluororesin and an electropolished metal material.

5. The purification device according to claim 4,
wherein in a case where the infill is coated with the fluororesin, and the fluororesin forms a coating layer, a water contact angle on an outermost surface of the coating layer is equal to or greater than 90°, or
in a case where the infill is formed of the fluororesin, a water contact angle on an outermost surface of the infill is equal to or greater than 90°.

6. The purification device according to claim 4,
wherein in a case where the infill is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the infill is formed of the electropolished metal material, and the metal material contains chromium and iron, a mass ratio of chromium atoms contained in a surface of the infill to iron atoms contained in the surface of the infill is 0.80 to 3.0.

7. A purification method of a chemical liquid, comprising:
a step of obtaining a purified product by distilling a chemical liquid by using the purification device according to claim 1.

8. A manufacturing device for manufacturing a chemical liquid, comprising:
a reaction portion for obtaining a reactant, which is a chemical liquid, by reacting a raw material;
a distillation column for obtaining a purified product by distilling the reactant; and
a first transfer pipeline which connects the reaction portion and the distillation column to each other so as to transfer the reactant to the distillation column from the reaction portion,
wherein an interior wall of the distillation column is coated with or formed of at least one kind of material selected from the group consisting of a fluororesin and an electropolished metal material,
the metal material contains at least one kind of metal selected from the group consisting of chromium and nickel, and
a total content of the chromium and the nickel is greater than 25% by mass with respect to a total mass of the metal material.

9. The manufacturing device according to claim 8,
wherein in a case where the interior wall of the distillation column is coated with the fluororesin, and the fluororesin forms a coating layer, a water contact angle on an outermost surface of the coating layer is equal to or greater than 90°, or
in a case where the interior wall of the distillation column is formed of the fluororesin, a water contact angle on an outermost surface of the interior wall of the distillation column is equal to or greater than 90°.

10. The manufacturing device according to claim 8,
wherein in a case where the interior wall of the distillation column is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the interior wall of the distillation column is formed of the electropolished metal material, and the metal material contains chromium and iron, a mass ratio of chromium atoms contained in a surface of the interior wall of the distillation column to iron atoms contained in the surface of the interior wall of the distillation column is 0.80 to 3.0.

11. The manufacturing device according to claim 8,
wherein an interior wall of the first transfer pipeline is coated with or formed of at least one kind of material selected from the group consisting of a fluororesin and an electropolished metal material.

12. The manufacturing device according to claim 11,
wherein in a case where the interior wall of the first transfer pipeline is coated with the fluororesin, and the fluororesin forms a coating layer, a water contact angle on an outermost surface of the coating layer is equal to or greater than 90°, or
in a case where the interior wall of the first transfer pipeline is formed of the fluororesin, a water contact angle on an outermost surface of the interior wall of the first transfer pipeline is equal to or greater than 90°.

13. The manufacturing device according to claim 11,
wherein in a case where the interior wall of the first transfer pipeline is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the interior wall of the first transfer pipeline is formed of the electropolished metal material, and the metal material contains chromium and iron, a mass ratio of chromium atoms contained in a surface of the interior wall of the first transfer pipeline to iron atoms contained in the surface of the interior wall of the first transfer pipeline is 0.80 to 3.0.

14. The manufacturing device according to claim 8, further comprising:
a filling portion for filling a container with the purified product; and
a second transfer pipeline which connects the distillation column and the filling portion to each other so as to transfer the purified product to the filling portion from the distillation column.

15. The manufacturing device according to claim 14,
wherein an interior wall of the second transfer pipeline is coated with or formed of at least one kind of material selected from the group consisting of a fluororesin and an electropolished metal material.

16. The manufacturing device according to claim 15,
wherein in a case where the interior wall of the second transfer pipeline is coated with the fluororesin, and the fluororesin forms a coating layer, a water contact angle on an outermost surface of the coating layer is equal to or greater than 90°, or
in a case where the interior wall of the second transfer pipeline is formed of the fluororesin, a water contact angle on an outermost surface of the interior wall of the second transfer pipeline is equal to or greater than 90°.

17. The manufacturing device according to claim 15,
wherein in a case where the interior wall of the second transfer pipeline is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the interior wall of the second transfer pipeline is formed of the electropolished metal material, and the metal material contains chromium and iron, a mass ratio of chromium atoms contained in a surface of the interior wall of the second transfer pipeline to iron atoms contained in the surface of the interior wall of the second transfer pipeline is 0.80 to 3.0.

18. The manufacturing device according to claim 14, further comprising:
a filter portion which is disposed in the middle of the second transfer pipeline so as to filter the purified product by using a filter.

19. The manufacturing device according to claim 8,
wherein an infill is disposed in the interior of the distillation column, and
the infill is coated with or formed of at least one kind of material selected from the group consisting of a fluororesin and an electropolished metal material.

20. The manufacturing device according to claim 19,
wherein in a case where the infill is coated with the fluororesin, and the fluororesin forms a coating layer, a water contact angle on an outermost surface of the coating layer is equal to or greater than 90°, or
in a case where the infill is formed of the fluororesin, a water contact angle on an outermost surface of the infill is equal to or greater than 90°.

21. The manufacturing device according to claim 19,
wherein in a case where the infill is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the infill is formed of the electropolished metal material, and the metal material contains chromium and iron, a mass ratio of chromium atoms contained in a surface of the infill to iron atoms contained in the surface of the infill is 0.80 to 3.0.

22. The manufacturing device according to claim 8,
wherein the reaction portion includes a reactor to which the raw material is supplied and in which a reaction proceeds, and
an interior wall of the reactor is coated with or formed of at least one kind of material selected from the group consisting of a fluororesin and an electropolished metal material.

23. The manufacturing device according to claim 22,
wherein in a case where the interior wall of the reactor is coated with the fluororesin, and the fluororesin forms a coating layer, a water contact angle on an outermost surface of the coating layer is equal to or greater than 90°, or
in a case where the interior wall of the reactor is formed of the fluororesin, a water contact angle on an outermost surface of the interior wall of the reactor is equal to or greater than 90°.

24. The manufacturing device according to claim 22,
wherein in a case where the interior wall of the reactor is coated with the electropolished metal material, and the metal material forms a coating layer and contains chromium and iron, a mass ratio of chromium atoms contained in a surface of the coating layer to iron atoms contained in the surface of the coating layer is 0.80 to 3.0, or
in a case where the interior wall of the reactor is formed of the electropolished metal material, and the metal material contains chromium and iron, a mass ratio of chromium atoms contained in a surface of the interior wall of the reactor to iron atoms contained in the surface of the interior wall of the reactor is 0.80 to 3.0.

25. A manufacturing method of a chemical liquid, comprising:
a reaction step of obtaining a reactant, which is a chemical liquid, by reacting a raw material; and
a purification step of obtaining a purified product by distilling the reactant by using a distillation column,
wherein an interior wall of the distillation column is coated with or formed of at least one kind of material selected from the group consisting of a fluororesin and an electropolished metal material,
the metal material contains at least one kind of metal selected from the group consisting of chromium and nickel, and
a total content of the chromium and the nickel is greater than 25% by mass with respect to a total mass of the metal material.

26. The manufacturing method of a chemical liquid according to claim 25,
wherein in a case where the interior wall of the distillation column is coated with the fluororesin, and the fluororesin forms a coating layer, a water contact angle on an outermost surface of the coating layer is equal to or greater than 90°, or
in a case where the interior wall of the distillation column is formed of the fluororesin, a water contact angle on an outermost surface of the interior wall of the distillation column is equal to or greater than 90°.

27. The manufacturing method of a chemical liquid according to claim 25,
wherein in a case where the interior wall of the distillation column is electropolished, includes a coating layer formed of the metal material, and the metal material contains chromium and iron, a mass ratio of chromium atoms contained in a surface of the coating layer to iron atoms contained in the surface of the coating layer is 0.80 to 3.0, or
in a case where the interior wall of the distillation column is formed of the electropolished metal material, a mass ratio of chromium atoms contained in a surface of the interior wall of the distillation column to iron atoms contained in the surface of the interior wall of the distillation column is 0.80 to 3.0.

28. The manufacturing method of a chemical liquid according to claim 25, further comprising:
a filling step of filling a container with the purified product after the purification step.

29. The manufacturing method of a chemical liquid according to claim 25, further comprising:
a filtering step of filtering the purified product by using a filter after the purification step.

30. The manufacturing method of a chemical liquid according to claim 29,
wherein the filter is formed of at least one kind of material selected from the group consisting of nylon, polypropylene, polyethylene, polytetrafluoroethylene, and a tetrafluoroethylene-perfluoroalkyl vinyl ether copolymer.

31. The manufacturing method of a chemical liquid according to claim 29,
wherein in the filtering step, the purified product is filtered plural times by using different kinds of filters.

32. The manufacturing method of a chemical liquid according to claim 29, further comprising:
a filling step of filling a container with the purified product after the filtering step.

33. The manufacturing method of a chemical liquid according to claim 25,
wherein the chemical liquid is used as at least one kind of agent selected from the group consisting of a pre-wet liquid, a developer, and a rinsing liquid for manufacturing a semiconductor.

34. A container for storing a chemical liquid,
wherein an interior wall of the container is coated with or formed of at least one kind of material selected from the group consisting of a polyolefin resin, a fluororesin, a metal material, and an electropolished metal material,
the metal material contains at least one kind of metal selected from the group consisting of chromium and nickel, and
a total content of the chromium and the nickel is greater than 25% by mass with respect to a total mass of the metal material.

35. The container according to claim 34,
wherein in a case where the interior wall of the container is coated with at least one kind of resin material selected from the group consisting of a polyolefin resin and a fluororesin, and the resin material forms a coating layer, a water contact angle on an outermost surface of the coating layer is equal to or greater than 90°, or
in a case where the interior wall of the container is formed of the resin material, a water contact angle on an outermost surface of the interior wall of the container is equal to or greater than 90°.

36. The container according to claim 34,
wherein the material is the electropolished metal material.

37. The container according to claim 34,
wherein in a case where the metal material contains chromium and iron, a mass ratio of chromium atoms contained in a surface of the interior wall of the container to iron atoms contained in the surface of the interior wall of the container is 0.80 to 3.0.

38. A chemical liquid storage container comprising:
the container according to claim 34; and
a chemical liquid stored in the container.

39. The chemical liquid storage container according to claim 38,
wherein the chemical liquid contains metal components containing at least one kind of element selected from the group consisting of Al, Ca, Cr, Co, Cu, Fe, Pb, Li, Mg, Mn, Ni, K, Ag, Na, Ti, and Zn, and
a content of metal particles, which contain the element, among the metal components is equal to or smaller than 100 mass ppt with respect to a total mass of the chemical liquid.

40. The chemical liquid storage container according to claim 38,
wherein the chemical liquid contains metal components containing at least one kind of element selected from the group consisting of Na, K, Ca, Fe, Cr, Ti, and Ni, and
a content of metal particles, which contain the element, among the metal components is equal to or smaller than 50 mass ppt with respect to a total mass of the chemical liquid.

41. The chemical liquid storage container according to claim 39,
wherein the content of the metal particles is equal to or smaller than 10 mass ppt with respect to the total mass of the chemical liquid.

42. The chemical liquid storage container according to claim 38,
wherein the chemical liquid contains metal components containing Fe, and
a content of metal particles, which contain the Fe, among the metal components is equal to or smaller than 10 mass ppt with respect to the total mass of the chemical liquid.

43. The manufacturing method of a chemical liquid according to claim 28,
wherein in the filling step, a container is filled with the purified product,
wherein an interior wall of the container is coated with or formed of at least one kind of material selected from the group consisting of a polyolefin resin, a fluororesin, a metal material, and an electropolished metal material,
the metal material contains at least one kind of metal selected from the group consisting of chromium and nickel, and
a total content of the chromium and the nickel is greater than 25% by mass with respect to a total mass of the metal material.

44. The manufacturing method of a chemical liquid according to claim 43, further comprising:
a step of washing the interior wall of the container by using a washing liquid before the filling step,
wherein a contact angle of the washing liquid with respect to the interior wall is 10° to 120°.

45. The manufacturing method of a chemical liquid according to claim 44,
wherein the chemical liquid contains at least one kind of component selected from the group consisting of water and an organic solvent, and
the washing liquid is at least one kind of liquid selected from the group consisting of the chemical liquid, the organic solvent, the water, and a mixture of these.
